(19) United States
(12) Patent Application Publication
(10) Pub. No.: US 2025/0009900 A1
(43) Pub. Date: Jan. 9, 2025

(54) OXAZAPHOSPHORINE ANTIBODY DRUG CONJUGATES AND METHODS OF USE

(71) Applicant: LA Life Products, LLC, Katy, TX (US)

(72) Inventor: Hector Alila, Katy, TX (US)

(73) Assignee: LA Life Products, LLC, Katy, TX (US)

(21) Appl. No.: 18/759,535

(22) Filed: Jun. 28, 2024

Related U.S. Application Data
(60) Provisional application No. 63/524,400, filed on Jun. 30, 2023.

(51) Int. Cl. A61K 47/68; A61K 31/136; A61K 31/166; A61K 31/337; A61K 31/404; A61K 31/4184; A61K 31/436; A61K 31/4439; A61K 31/454; A61K 31/47; A61K 31/4745; A61K 31/495; A61K 31/502; A61K 31/5025; A61K 31/513; A61K 31/519; A61K 31/55; A61K 31/573; A61K 31/585; A61K 31/704; A61K 31/7068; A61K 38/09; A61K 38/19; A61K 38/20; A61K 38/21; A61K 39/395; A61K 45/06; A61K 51/00

(52) U.S. Cl. CPC A61K 47/6851; A61K 31/136; A61K 31/166; A61K 31/337; A61K 31/404; A61K 31/4184; A61K 31/436; A61K 31/4439; A61K 31/454; A61K 31/47; A61K 31/4745; A61K 31/495; A61K 31/502; A61K 31/5025; A61K 31/513; A61K 31/519; A61K 31/55; A61K 31/573; A61K 31/585; A61K 31/704; A61K 31/7068; A61K 38/09; A61K 38/191; A61K 38/193; A61K 38/2006; A61K 38/2013; A61K 38/204; A61K 38/2046; A61K 38/208; A61K 38/2086; A61K 38/212; A61K 38/217; A61K 39/3955; A61K 39/39558; A61K 45/06; A61K 47/6803; A61K 51/00

(57) ABSTRACT

Disclosed herein are antibody-drug conjugates (ADCs) comprising an antibody conjugated via a linker to an oxazaphosphorine drug moiety and methods of using the antibody drug conjugates. Included are methods of treating undesirable or aberrant cell proliferation or hyperproliferative disorders, such as non-metastatic and metastatic neoplasia, cancer, stem cells and malignancies that express targets that bind to such antibodies, or fragments antibodies, antibody heavy and light chains or nanobodies.

### CROSS-REFERENCE TO RELATED APPLICATIONS

This application claims the benefit of U.S. Provisional Patent Application No. 63/524,400, filed Jun. 30, 2023, which is incorporated by reference herein in its entirety.

### FIELD

The invention generally relates to cancer treatment. The invention specifically relates to antibody-drug conjugates (ADCs) for use in cancer immunotherapy.

### BACKGROUND

Human epidermal growth factor receptor-2 (HER2) is a well-established target across a variety of malignancies, for tumors with HER2 overexpression or amplification (e.g. breast cancer, gastric, colorectal, cervical, endometrial, bladder, esophagogastric, biliary tract carcinoma, salivary gland) and HER2 mutations (e.g. NSCLC and more than 20 other tumor types) Raghav K P S and Moasser M M, Clin Cancer Res. 2023 Jul. 5; 29 (13): 2351-2361. doi: 10.1158/1078-0432.CCR-22-0283. HER2 targeting antibodies and ADCs, most prominently trastuzumab, trastuzumab-deruxtecan (T-DXd) and Trastuzumab emtansine (T-DM1), are standards of care for HER2 breast and other tumor types and for HER2 mutant tumors e.g. non-small cell lung cancer (NSCLC). Despite the effectiveness of the ADCs, resistance still occurs in many patients. For example, resistance to T-DXd emerges and appears to be most commonly due to resistance to the topoisomerase inhibitor payload because the loss of HER2 expression occurs in only a minority of the resistant cases. Another approved ADC that uses a topoisomerase inhibitor is Sacituzumab govitecan that treats cancers such breast cancer, bladder cancer that overexpress trophoblast cell surface antigen 2 (TROP2). TROP2 is also expressed in a variety of malignancies e.g. cervical, colorectal, ovarian, prostate, thyroid, gastric, brain, esophageal, head and neck, pancreatic and endometrial cancers. Development of resistance to Sacituzumab govitecan also occurs in part due to resistance to the topoisomerase inhibitor payload. Therefore, there is a major unmet need for a HER2 and TROP2-targeting ADCs that use an alternative agent as a payload. This invention describes using oxazaphosphorines as alternative potent drug payloads to produce novel ADCs.

### SUMMARY

In one aspect, the subject matter described herein is directed to an antibody conjugated to an oxazaphosphorine drug via a linker having the formula:

Ab-(L-D)n

wherein, D is an oxazaphosphorine, covalently bound via a linker (L) to an antibody (Ab) or antibody fragment that binds to a tumor antigen, and n has a value of 2 to 20.

Another aspect of the subject matter described herein is the antibody may be engineered to enhance its antitumor cytotoxicity. The antibody may also be designed to bind simultaneously to one or more antigens on the target cells.

Another aspect of the subject matter described herein is an antibody fragment, nanobody, affibody or Heavy/Light chains.

Another aspect of the subject matter described herein is an activated oxazaphosphorine selected from a group comprising an active metabolite, analog or derivative that has direct antitumor cytotoxicity (“tumoricidal activity”).

Another aspect of the subject matter described herein is an activated oxazaphosphorine selected from a group comprising an active metabolite, analog or derivative that stimulates the antitumor immune system by depleting or reducing the number and/or inhibiting the function of immunosuppressive cells such as regulatory T cells (Tregs) in the tumor microenvironment (TME).

Another aspect of the subject matter described herein is a pharmaceutical composition comprising an oxazaphosphorine antibody-drug conjugate, and one or more pharmaceutically acceptable excipients.

Another aspect of the subject matter described herein is the use of an antibody-oxazaphosphorine drug conjugate in methods of treating patients by administering to a subject a pharmaceutical composition comprising the oxazaphosphorine-antibody conjugate.

Another aspect of the subject matter described herein is a method of making an antibody-drug conjugate covalently linked to an oxazaphosphorine.

Another aspect of the subject matter described herein is an article of manufacture comprising a pharmaceutical composition comprising an antibody or fragment conjugated to oxazaphosphorine, a container, and a package insert or label indicating that the pharmaceutical composition can be used to treat a patient.

In one aspect, the subject matter described herein is directed to a composition comprising an antibody-drug conjugate of the formula Ab-(L-D)ₙ wherein Ab is an antibody, antibody fragment, antibody chain, affibody, aptamer, or a nanobody; D is an active oxazaphosphorine payload; L is a linker; and n has a value of 2 to 20.

In another aspect, n has a value of 2-8.

In another aspect, the Ab binds to a tumor-associated antigen from at least one of a group comprising HER2, HER3, VEGF-A, VEGFR-2, CSF-1R, PD-L1, CEACAM5 or CEACAM6, ROR1, CD20, CD19, CD22, CD30, CD33, CD133, CD38, CD39 CD25, CD47, CD52, CD56, CD70, CD73, CD74, CD79b, CD155, CD166, FGF-receptor, B7-H3, B7-H4, LIV1, PSMA, PSCA, MAGE-A4, EpCAM, IL1R, CCR8, CCR4, Claudin, APPL2, BCMA, EGFR, DLL3/4, SSX-2, Tissue Factor, folate receptor, mesothelin receptor, NaPi2b, 5T4, Nectin-4, Nectin-2 (CD112), c-MeT, TROP2, Fibroblast Activation Protein (FAP), LHRH (GnRH) receptor, gonadotropin (LH/hCG, FSH) receptor, prolactin receptor, claudins; survivin, STEAP1, Transferrin receptor 1, NRG1, EphB2, and Caveolin-1.

In another aspect, the oxazaphosphorine payload is cytotoxic to T-regulatory cells.

In another aspect, the oxazaphosphorine payload is cytotoxic to a cancer cell.

In another aspect, the oxazaphosphorine payload is of the formula:

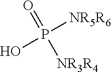

wherein at least one of R₃, R₄, R₅, and R₆ is a CH₂CH₂Y; wherein Y is a halogen; and wherein the remaining R₃, R₄, R₅, and R₆ groups are a hydrogen or a lower alkyl group.

In another aspect, the halogen is Cl or Br.

In another aspect, the hydrogen in R₃ and R₅ are replaced with deuterium to form CD₂CH₂ Y or methyl (CH₃) to form CH₃CH₂Y.

In another aspect, the oxazaphosphorine payload is of a structure selected from the group consisting of

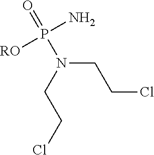

wherein R is an alkyl chain;

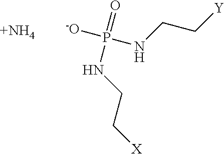

wherein X and Y are halogen leaving groups;

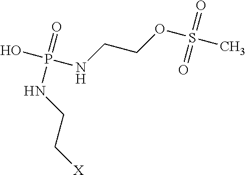

wherein X is a halogen leaving group; and

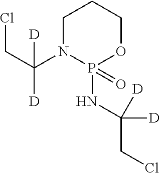

wherein D is deuterium.

In another aspect, the oxazaphosphorine payload is selected from 4-hydroxycyclophosphamide, aldophosphamide, phosphoramide mustard, 3-hydroxypropanal, isophosphoramide mustard, 4-hydroxycyclophosphamide, 4-hydroperoxycyclophosphamide, 4-hydroxyifosfamide, 4-hydroperoxyifosfamide evofosfamide, mafosfamide, glufosfamide, or trifosfamide mustard.

In another aspect, the oxazaphosphorine payload is phosphoramide mustard.

In another aspect, the oxazaphosphorine payload is selected from an analog or derivative of 4-hydroxycyclophosphamide, aldophosphamide, phosphoramide mustard, 3-hydroxypropanal, isophosphoramide mustard, 4-hydroxycyclophosphamide, 4-hydroperoxycyclophosphamide, 4-hydroxyifosfamide, 4-hydroperoxyifosfamide evofosfamide, mafosfamide, glufosfamide, or trifosfamide mustard.

In another aspect, the derivative of 4-hydroperoxyifosfamide is 4-hydroxyifosfamide.

In another aspect, the analog or derivative of 4-hydroperoxyifosfamide (4-HO-ifosfamide) is deuterated (d4-hydroxyifosfamide).

In another aspect, the 4-hydroperoxycyclophosphamide derivative is 4-hydroxycyclophosphamide.

In another aspect, the oxazaphosphorine payload is of the structure:

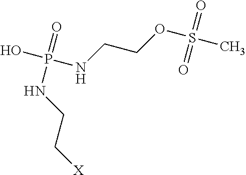

wherein X is Cl or Br.

In another aspect, X is Cl.

In another aspect, the oxazaphosphorine payload is of the structure:

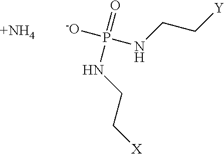

wherein X and Y represent independent leaving groups.

In another aspect, the oxazaphosphorine payload metabolite is isophosphoramide mustard or its analogs.

In another aspect, the oxazaphosphorine payload is selected from dimethyl-isophosphoramide mustard or its analogs or a 4-hydroxy-derivative (4-HO-ifosfamide) or its analog or derivative.

In another aspect, the oxazaphosphorine payload metabolite is selected from bromo-isophosphoramide mustard or its analog or derivative comprising evofosfamide or dimethyl-isophosphoramide mustard.

In another aspect, the oxazaphosphorine payload is geranyloxy-isophosphoramide mustard metabolite or its analog or derivative.

In another aspect, the oxazaphosphorine payload metabolite is mafosfamide or its analog or derivative.

In another aspect, the oxazaphosphorine payload metabolite is glufosfamide or its analog or derivative.

In another aspect, the oxazaphosphorine payload metabolite is triphosphoramide mustard or its analog or derivative.

In another aspect, the composition further comprises a therapeutic agent.

In another aspect, the composition further comprises an anti-TAM (tumor-associated macrophage) drug.

In another aspect, the composition further comprises one or more antibodies that enhance anti-tumor immunity selected from the group anti-PD-1, anti-PD-L1, anti-CTLA4, anti-LAG3, anti-GITR, anti-TIM-3, anti-TIGIT, anti-CD96, anti-CD226, anti-CD155, anti-CD47, anti-CEACAMI, anti-CEACAM5, anti-CEACAM6, anti-galectin-1, anti-claudin, anti-Siglec-15 antibodies, anti-VISTA, anti-CD137, anti-CCR4 antibody, anti-CCR8 antibody, anti-CD39 antibody, anti-CD25 antibody, anti-CD-73 antibody, and anti-CSFR1.

In another aspect, the composition further comprises one or more cytokines selected from IL-1B, IL-2, IL-6, IL-7, IL-12, IL-15, IL-21, IL-23, IL-27, TNFα, IFNα, IFNγ, GM-CSF, anti-IL2R, activators of Toll-like receptors (TLRs), and stimulators of interferon genes (STING).

In another aspect, the activators of Toll-like receptors are poly(I: C) and CpG.

In another aspect, the composition further comprises one or more chemotherapeutic drugs selected from the group 5-fluorouracil, 2′-deoxy-5-fluoridine, cytarabine, cladribine, fludarabine, pentostatine, gemcitabine, and 6-thioguanine, melphalan and any derivatives thereof; and an alkylating drug chlorambucil, bendamustine, melphalan, alkylating agents or anthracyclines such as doxorubicin, epirubicin, daunarubicin; temozolomide, oxaliplatin, cisplatin, chlorambucil, mechlorethamine, mitoxantrone, pexidartinib, lenvatinib, trabectedin, HDAC inhibitors, anti-angiogenic drugs, bisphosphonates, taxane, vinorelbine, ibrutinib, cribulin, resiquimod, gardiquimod or their analogs, anti-semaphorin 4D, CXCR2 blockers, axitinib, sorafenib, carbozantinib, sunitinib, multi-kinase inhibitor e.g. regorafenib, bifunctional PROTACs and molecular glue degraders (e.g. thalidomide, lenalidomide, pomalidomide, avadomide), vandetanib, cediranib or their analogs, anti-VEGF-A antibody (bevacizumab), anti-VEGF-R₂ antibody (ramucirumab), TRL9 agonists, anti-CCR4 antibody, PPARγ agonists, miRNA, angiotensin receptor blockers, CXCR4 blockers, CD4/6 inhibitors, proteosome inhibitors, JAK1/2 inhibitors, Bruton Kinase (BTK) inhibitors, kinase inhibitors, topoisomerase inhibitors, epigenetic inhibitors, DNMT, HMT, HDM inhibitors, PARP-inhibitors, hormone antagonists, anti-prolactin, VEGI, osteopontin, maspin, canstatin, itraconazole, carboxyamidotriazole, suramin, thrombospondin, tetrathiomolybdate, linomide, tasquinimod, carfilzomib, sunitinib, pazobanib, everolimus; anti-hormones: luteinizing hormone releasing hormone (LHRH) antagonists, tamoxifen, cortisol analogs, steroid receptor modulators or antagonists, cancer metabolism inhibitors, radioisotope, radiopharmaceutical, vinca alkaloids, mTOR inhibitors, MEK-inhibitors, BRAF inhibitors, MAPK and tyrosine kinase inhibitors, bortezomib, demethylating agent, bleomycin, alkylating agent, dacarbazine, temozolomide, CELLMODs and targeted protein degrader.

In another aspect, the taxane is selected from the group consisting of docetaxel, paclitaxel, cabazitaxel, and 6-α-hydroxypaclitaxel.

In another aspect, the epigenetic inhibitor is directed to HDAC, DNMT, LSD1, DOTIL, BET, or EZH.

In another aspect, the hormone antagonist is lupron.

In another aspect, the PARP-inhibitors are selected from the group consisting of 1-aminobenzamide, iniparib, BMN-573, olaparib, niraparib, talazoparib, rucaparib, veliparib, CEP 9722, MK 4827, BGB-290, and derivatives thereof.

In another aspect, the cortisol analogs are selected from the group consisting of prednisone, dexamethasone; raloxifene, anastrozole, letrozole, exemestane, spironolactone, cyproterone acetate, bicalutamide, RU53063, thiohydantoin, RD162, and any of their derivatives thereof.

In another aspect, the steroid receptor modulators or antagonists are selected from the group consisting of anti-estrogens, anti-progestins, anti-androgens, anti-corticoids, and anti-thyroid hormone.

In another aspect, the cancer metabolism inhibitors are selected from the group consisting of pyruvate kinase inhibitors and isocitrate dehydrogenase inhibitors.

In another aspect, the radioisotope is radium Ra 223 dichloride, lutetium Lu 177, Actinium 225, Yttrium 90, Technetium 99, or iodine 131.

In another aspect, the vinca alkaloids are selected from the group consisting of vinblastine, vincristine, vindesine, and vinorelbine, and any derivatives thereof.

In another aspect, the protein degrader is a proteolysis-targeting chimeras (PROTACs) or a molecular glue.

In another aspect, the composition further comprises a tumor targeting antibody.

In another aspect, the composition further comprises a cell therapy.

In another aspect, the composition further comprises a gene therapy.

In another aspect, the composition further comprises a cancer vaccine.

In another aspect, the composition further comprises an oncolytic virus.

In another aspect, the antibody is trastuzumab.

In another aspect, the antibody is sacituzumab.

In another aspect, the linker and active oxazaphosphorine payload is RTX5007.

In another aspect, the antibody-drug conjugate is trastuzumab-RTX5007.

In another aspect, the antibody-drug conjugate is sacituzumab-RTX5007.

In another aspect, the oxazaphosphorine payload is isophosphoramide mutard.

In one aspect, the subject matter described herein is directed to a method for treating cancer in a subject in need thereof comprising administering to the subject a composition comprising an antibody-drug conjugate of the formula Ab-(L-D)n wherein Ab is an antibody, antibody fragment, antibody chain, affibody, aptamer, or a nanobody; D is an active oxazaphosphorine payload; L is a linker; and n has a value of 2 to 20.

In another aspect, the antibody-drug conjugate is present in a pharmaceutical composition comprising one or more pharmaceutically acceptable carriers.

In another aspect, the method further comprises administering a therapeutic agent.

In another aspect, the method further comprises administering chemoradiation.

In another aspect, the method further comprises administering an anti-TAM (tumor-associated macrophage) drug.

In another aspect, the method further comprises administering one or more antibody that enhances anti-tumor immunity selected from the group anti-PD-1, anti-PD-L1, anti-CTLA4, anti-LAG3, anti-GITR, anti-TIM-3, anti-TIGIT, anti-CD96, anti-CD226, anti-CD155, anti-CD47, anti-CEACAMI, anti-CEACAM5, anti-CEACAM6, anti-galectin-1, anti-Siglec-15 antibodies, anti-VISTA, anti-CD137, anti-CCR4 antibody, anti-CD39 antibody, anti-CD25 antibody, anti-CD-73 antibody, and anti-CSFR1.

In another aspect, the method further comprises administering one or more cytokines from the group IL-1β, IL-2, IL-6, IL-7, IL-12, IL-15, IL-21, IL-23, IL-27, TNFα, IFNα, IFNγ, GM-CSF), anti-IL2R, activators of Toll-like receptors (TLRs), and stimulators of interferon genes (STING).

In another aspect, the activators of Toll-like receptors are poly(I: C) and CpG.

In another aspect, the method further comprises one or more chemotherapeutic drugs selected from the group 5-fluorouracil, 2′-deoxy-5-fluoridine, cytarabine, cladribine, fludarabine, pentostatine, gemcitabine, and 6-thioguanine, melphalan and any derivatives thereof; and an alkylating drug chlorambucil, bendamustine, melphalan, alkylating agents or anthracyclines such as doxorubicin, epirubicin, daunarubicin; temozolomide, melphalan, oxaliplatin, cisplatin, chlorambucil, mechlorethamine, mitoxantrone, pexidartinib, lenvatinib, trabectedin, HDAC inhibitors, anti-angiogenic drugs, bisphosphonates, taxane, vinorelbine, ibrutinib, cribulin, resiquimod, gardiquimod or their analogs, anti-VISTA antibody, anti-semaphorin 4D, CXCR2 blockers, axitinib, sorafenib, carbozantinib, sunitinib, regorafenib, thalidomide, lenalidomide, pomalidomide, avadomide, vandetanib, cediranib or their analogs, anti-VEGF-A antibody (bevacizumab), anti-VEGF-R₂ antibody (ramucirumab), TRL9 agonists, PPARγ agonists, miRNA, angiotensin receptor blockers, CXCR4 blockers, CD4/6 inhibitors, proteosome inhibitors, JAK1/2 inhibitors, Bruton Kinase (BTK) inhibitors, kinase inhibitors, topoisomerase inhibitors, epigenetic inhibitors, DNMT, HMT, HDM inhibitors, PARP-inhibitors, hormone antagonists, anti-prolactin, VEGI, osteopontin, maspin, canstatin, itraconazole, carboxyamidotriazole, suramin, thrombospondin, tetrathiomolybdate, linomide, tasquinimod, carfilzomib, sunitinib, pazobanib, everolimus; anti-hormones: luteinizing hormone releasing hormone (LHRH) antagonists, tamoxifen, cortisol analogs, steroid receptor modulators or antagonists, cancer metabolism inhibitors, radioisotope, radiopharmaceutical, vinca alkaloids, mTOR inhibitors, MEK-inhibitors, BRAF inhibitors, MAPK and tyrosine kinase inhibitors, bortezomib, demethylating agent, bleomycin, alkylating agent, dacarbazine, temozolomide, and CELLMODs.

In another aspect, the taxane is selected from the group docetaxel, paclitaxel, cabazitaxel, and 6-α-hydroxypaclitaxel.

In another aspect, the epigenetic inhibitor is directed to HDAC, DNMT, LSD1, DOTIL, BET, or EZH.

In another aspect, the hormone antagonist is lupron.

In another aspect, the PARP-inhibitors are selected from 1-aminobenzamide, iniparib, BMN-573, olaparib, niraparib, talazoparib, rucaparib, veliparib, CEP 9722, MK 4827, BGB-290, and derivatives thereof.

In another aspect, the cortisol analogs are selected from the group predisone, dexamethasone; raloxifene, anastrozole, letrozole, exemestane, spironolactone, cyproterone acetate, bicalutamide, RU53063, thiohydantoin, RD162, and any of their derivatives thereof.

In another aspect, the steroid receptor modulators or antagonists are selected from the group consisting anti-estrogens, anti-progestins, anti-androgens, anti-corticoids, and anti-thyroid hormone.

In another aspect, the cancer metabolism inhibitors are selected from the group consisting of pyruvate kinase inhibitors and isocitrate dehydrogenase inhibitors.

In another aspect, the radioisotope is radium Ra 223 dichloride, lutetium Lu 177, Actinium 225, Yttrium 90, Technetium 99, or iodine 131.

In another aspect, the vinca alkaloids are selected from the group consisting of vinblastine, vincristine, vindesine, and vinorelbine, and any derivatives thereof.

In another aspect, the method further comprises administering a tumor targeting antibody.

In another aspect, the method further comprises administering a cell therapy.

In another aspect, the method further comprises administering a gene therapy.

In another aspect, the method further comprises administering a cancer vaccine.

In another aspect, the method further comprises administering radiotherapy.

In another aspect, the method further comprises administering phototherapy.

In another aspect, the method further comprises administering an oncolytic virus.

In another aspect, the antibody is trastuzumab.

In another aspect, the antibody is sacituzumab.

In another aspect, the linker and active oxazaphosphorine payload is RTX5007.

In another aspect, the antibody-drug conjugate is trastuzumab-RTX5007. the antibody-drug conjugate is sacituzumab-RTX5007.

### BRIEF DESCRIPTION OF THE DRAWINGS

The present technology will be better understood on reading the following detailed description of non-limiting embodiments thereof, and on examining the accompanying drawings in which:

FIG. 1 depicts Oxazaphosphorines and Metabolism of Ifosfamide (from Skarbek C. et al. 2015. J. Med. Chem. 58:705-717);

FIG. 2 depicts conjugation of Trastuzumab and an oxazaphosphorine payload THLP-1 (SEC): 101921-10P;

FIG. 3 depicts Drug to Antibody Ratio (DAR) and concentration of 101921-10P determined by HIC and UV. DAR calculation From UV: DAR=(ε p252 nm-ε p280 nm R)/(ε d280nmR-ε d252 nm) (See ref: Clinical Cancer Research: 20Apr. 10, 7063˜7070);

FIG. 4 depicts an example of a synthesis of linker payload;

FIG. 5 depicts Tregs in the tumor microenvironment and the pivotal role they play in suppressing antitumor immunity (Dees, et al., Eur. J. Immunol. 2021. 51:280-291);

FIG. 6 depicts the effects of Treg depletion on other immunosuppressive cells in TME (modified from Dees, et al., Eur. J. Immunol. 2021. 51:280-291); and

FIG. 7 depicts Treg depletion and cancer cell cytotoxicity in the TME; and

FIG. 8 depicts examples of oxazaphosphorine structures and biotransformation pathways from Anderson et al., Cancer Control: Journal of the Moffitt Cancer Center. January 2008.

FIG. 9 depicts cyclosphosphamide metabolism. Gor et al. Breast Cancer Research 2010, 12: R26.

FIG. 10 depicts synthesis of RTX5007;

FIG. 11 depicts synthesis of RTX5014;

FIG. 12 depicts synthesis of RTX5015;

FIG. 13 depicts monomer determination of Herceptin-RTX-5007 by SEC;

FIG. 14A-14B depict the drug-to-antibody ratio (DAR) determination for Herceptin-RTX-5007 by LC-MS;

FIG. 15 depicts determination of the concentration of Herceptin-RTX-5007 by measuring absorbance at 280 nanometers on a nanodrop 2000C E1% with 14.8 extinction coefficient;

FIG. 16 depicts monomer determination for Sacituzumab-RTX-5007 by SEC;

FIG. 17A-17B depict DAR determination for Sacituzumab-RTX-5007 by LC-MS;

FIG. 18 depicts determination of the concentration of Sacituzumab-RTX-5007 by measuring absorbance at 280 nanometers on a nanodrop 2000C E1% with 15.6 extinction coefficient;

FIG. 19A-19B depict the binding kinetics of Herceptin to HER2; FIG. 19B depicts the individual concentration dependent calculations (Local fit);

FIG. 20A-20B depict the binding kinetics of Herceptin-ADC to HER2; FIG. 20B depicts the individual concentration dependent calculations (Local fit);

FIG. 21A-21B depict the binding kinetics of Isotype to HER2; FIG. 21B depicts the individual concentration dependent calculations (Local fit);

FIG. 22A-22B depict the binding kinetics of Sacituzumab to TROP2; FIG. 22B depicts the individual concentration dependent calculations (Local fit);

FIG. 23A-23B depict the binding kinetics of Sacituzumab-ADC to TROP2 Binding kinetics of Sacituzumab-ADC to TROP2; FIG. 23B depicts the individual concentration dependent calculations (Local fit);

FIG. 24A-24B depict the binding kinetics of Isotype to TROP2; FIG. 24B depicts the individual concentration dependent calculations (Local fit);

FIG. 25A-25B depicts live cell image analysis (IncuCyte S3) assessment of NCI-N87 cells across a range of seeding densities (FIG. 25A low density; FIG. 25B high density) in 96-well plates to monitor cell growth for up to 120 Hrs;

FIG. 26A depicts N87 cells treated with Herceptin-ADC (NJBP-0165-129) across dose levels ranging from 30 mg/mL to 0.03 mg/mL in the presence of Caspase 3/7 and imaged every six hours on phase contrast to monitor % confluence and 488 nm fluorescence to monitor apoptosis, FIG. 26B-26E depict IC50 curves plotted for 24 (26B), 48 (26C), 72 (26D), and 96 (26E) hours of culture;

FIG. 27A depicts N87 cells treated with Sacituzumab-ADC (NJBP-0165-126) across dose levels ranging from 30 mg/mL to 0.03 mg/mL in the presence of Caspase 3/7 and imaged every six hours on phase contrast to monitor % confluence and 488 nm fluorescence to monitor apoptosis; FIG. 27B-27E depict IC50 curves plotted for 24 (27B), 48 (27C), 72 (27D), and 96 (27E) hours of culture.

### DETAILED DESCRIPTION

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it is intended that the particular value forms another embodiment.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a.” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

The term “active oxazaphosphorine payload” means that the oxazaphosphorine is not a prodrug and does not require metabolization to be active.

The abbreviation IPM stands for isophosphoramide mustard and the abbreviation PM stands for phosphoramide mustard.

The drug-to-antibody ratio (DAR) is the average number of drug molecules conjugated to an antibody.

Cancer continues to be a leading cause of death worldwide, accounting for nearly 10 million deaths in 2020, which is nearly one in six deaths. According to American Cancer Society, by 2040, the global burden is expected to grow to 27.5 million new cancer cases and 16.3 million cancer deaths simply due to the growth and aging of the population.

There have been advances in novel cancer therapies such as targeted chemotherapies, immunotherapies such as immune checkpoint inhibitors (ICIs, cell therapies, and antibody-drug conjugates (ADCs) that have revamped cancer care and improved survival rates for patients. However, there is a need to develop newer treatments considering that the mortality rate is still too high and a majority of patients do not respond or develop resistance to current treatments and some patients cannot tolerate the treatments due to toxicities.

Antibody drug conjugates (ADCs) are targeted chemotherapeutic molecules that combine the properties of both antibodies and cytotoxic drugs to deliver toxic agents to antigen-expressing tumor cells. They are typically designed to bind to the target cancer cells and release of the toxic agent may require internalization. Release of the drug occurs after cleavage of the linker by enzymes or low pH inside the cancer cell. Alternatively, for ADCs that are not internalized, the toxic agent is released by cleavage of a linker that is sensitive to hypoxic or low pH within the tumor microenvironment (TME).

To date, the ADCs used in the clinic consist of targeted monoclonal antibodies linked to super-toxic agents such as calicheamicins, auristatins, maytansinoids, or pyrollobenzodiazepines. These agents are cytotoxic at very low (nanomolar or picomolar) concentrations. After delivery to the tumor, these highly toxic payloads re-enter the circulation and distribute to other organs where they cause serious toxicities such as cardiac toxicity, neutropenia, anemia, fatigue, diarrhea, neurotoxicity, blindness, nephrotoxicity, or even death. Consequently, these ADCs have a “black box” warning on their labels. A black box warning is the most severe warning imposed by the FDA on a prescription drug because the drug can be potentially fatal, life-threatening, or can cause disabling adverse effects to the patient. This means that such drugs must be restricted and must be closely monitored during and after administration. The effectiveness of immunotherapy depends on the conditions within the TME, including the preponderance of immunosuppressive cells. Poor prognosis is associated with increased infiltration of immunosuppressive cells within the TME.

A current problem in cancer immunotherapy is that immunosuppressive regulatory T cells (Tregs) within the TME are a major obstacle to developing effective immunotherapy for cancer patients. Current methods to deplete Tregs lack distribution specificity because they also deplete Tregs in blood circulation and disrupt systemic immune homeostasis resulting in autoimmunity, systemic inflammation, and off-target toxicities.

The current disclosure describes a method to selectively deplete Tregs within the tumor microenvironment (TME) without disruption of systemic Treg immune homeostasis using ADCs that deliver oxazaphosphorine directly into the TME. Oxazaphosphorine metabolites are known to induce antitumor immunity by selective toxicity against Tregs (Eid RA et al, 2016, Cancer Immunol Res; 4 (5); 377-82. doi: 10.1158/2326-6066.CIR-16-0048; Traverso I et al. Human Immunology 73 (2012) 207-213, doi: 10.1016/j.humimm.2011.12.020; Voelcker G 2018, Anti-Cancer Drugs 29:411-415; Heylmann D, Bauer M, Becker H, van Gool S, Bacher N, et al. (2013), PLoS ONE 8 (12): c83384. doi: 10.1371/journal.pone.0083384). The ADC target and bind to cancer cells to release a therapeutic oxazaphosphorine payload that depletes Tregs within the TME. Dual killing of cancer cells and Tregs triggers immune memory for prolonged antitumor immunity. The therapeutic index is improved by increasing local delivery of therapeutic payload within the TME to minimize systemic toxicities and re-energizes exhausted T cells to overcome resistance to immunotherapy.

This invention describes ADCs that can provide better safety profiles than the approved ADCs because the drug payloads used are not as super-toxic and resistant cancer cells respond to oxazaphosphorine payloads described above.

The invention relates to ADCs comprising of a tumor-targeting antibody or antibody fragment conjugated via a linker to an oxazaphosphorine payload. ADCs comprising antibodies conjugated to active oxazaphosphorines have not heretofore been made.

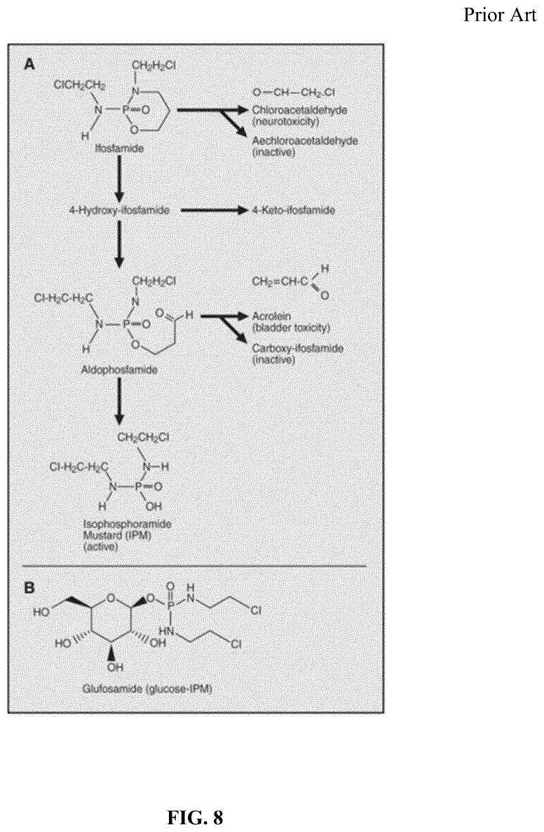

Oxazaphosphorines include, but are not limited to, cyclophosphamide, ifosfamide, and trofosfamide. Oxazaphosphorine derivatives include, but are not limited to, mafosfamide, glufosfamide, β-D-glucosylisophosphoramide mustard, aldophosphamide perhydrothiazine, and aldophosphamide thiazolidine. Cyclophosphamide and ifosfamide are prodrugs are require activation by hepatic cytochrome P450. Zhang, et al. (2005) Drug Metabolism Reviews, 37:4, 611-703. 4-hydroxy cyclophosphamide is a primary metabolite of cyclophosphamide. FIG. 8 depicts oxazaphosphorine structures and biotransformation pathways from Anderson et al., Cancer Control: Journal of the Moffitt Cancer Center · January 2008.

Oxazaphosphorines are alkylating agents that have been used in routine clinical practice for treatment of cancer for decades. They are prodrugs that require cytochrome p450 bioactivation in the liver leading to 4-hydroxy derivatives which are transported to the tumor through the blood circulation. They have tumoricidal and immunostimulatory properties. They kill cancer cells directly by causing immunogenic cell death (ICD). Their antitumor immune stimulatory properties occur because they selectively kill immunosuppressive cells such as regulatory T cells (Tregs) leading to reactivation and proliferation of tumor-specific cytotoxic T cells, NK cells, and macrophages that contribute to killing the cancer cells. Upon binding to a tumor, the antibody moiety of the ADC also kills cancer cells directly. The localized delivery and release of the Oxazaphosphorine payload within the tumor reduces the systemic exposure, which is essential for minimizing or reducing systemic toxicity and improves safety and tolerability to the patient.

The ADC can also be combined with established standard of care therapies such as immune checkpoint inhibitors, chemotherapy, radiotherapy, phototherapy, gene therapy, or cell therapy to maximize therapeutic benefits to the patient.

Unlike previous ADCs that require highly potent toxic drug payloads, low doses (concentrations) of Oxazaphosphorines are sufficient to exert their immunomodulatory effects. For example, it is well known that administering low doses of cyclophosphamide to patients at regular intervals (metronomic dosing) is sufficient to stimulate the immune system by depletion or reduction of immunosuppressive regulatory T cells (Tregs). Nonetheless, systemic toxicity still occurs because oral or systemic administration results in distribution and exposure of normal tissues in the body. Relative to the potent payloads used in other ADCs, any toxicity of oxazaphosphorines would be predictable and manageable based on decades of clinical experience.

Immune checkpoint inhibitors (ICIs) have also revolutionized cancer treatment. However, only 15-25% of patients respond to ICI therapy, while a large proportion experience systemic immune-related toxicities such as autoimmune disease, cardiopulmonary complications, and a majority of the patients eventually develop resistance. Oxazaphosphorine chemotherapeutic agents, such as cyclophosphamide, can be promising partners for use with immune checkpoint inhibitors because they synergize to improve anticancer immunity (George M et al., Cancer Res (2022) 82 (12_Supplement): 4167. https://doi.org/10.1158/1538-7445.AM2022-4167). Combination of cyclophosphamide with anti-CTLA-4 or anti-PD-1 antibody was shown to elicit potent tumor control in a mouse melanoma model. In the clinic, combination of an ifosfamide derivative, evofosfamide, with ipilimumab, an anti-CTLA-4, demonstrated good clinical activity in 15 of 22 patients, but drug-related toxicity was common (Hegde A, Jayaprakash P, Couillault C A, Piha-Paul S, Karp D, Rodon J, et al., Clin Cancer Res 2021; 27:3050-60). These results show that although synergy is achieved by combining an Oxazaphosphorine with a checkpoint inhibitor, dose-limiting toxicity caused by systemic exposure can be improved by localizing the effects within the tumor microenvironment.

The main mechanism responsible for development of resistance to cancer treatment with immune checkpoint inhibitors (ICIs) is the infiltration of immunosuppressive cells such as T regulatory cells (Tregs) into the tumor microenvironment (TME). Tregs are a subset of CD4-positive T cells that play a central role in suppressing antitumor immunity by facilitating tumor cells to escape immune surveillance (Lee, G. R., Phenotypic and functional properties of tumor-infiltrating regulatory T cells. Mediators Inflamm. 2017. 2017:5458178). They are the predominant immunosuppressive cells present within the tumor microenvironment (TME), accounting for 10-50% of CD4 T cells residing within human tumors (Togashi, Y., Shitara, K. and Nishikawa, H., Regulatory T cells in cancer immunosuppression-implications for anticancer therapy. Nat. Rev. Clin. Oncol. 2019. 16:356-371). Indeed, high frequency of infiltrating Tregs in the TME is associated with poor prognosis and low survival rates in various types of solid tumors such as breast, prostate, lung, colorectal, ovarian, cervical, bladder, renal, pancreatic, hepatocellular, skin, brain, head and neck cancers (Tanaka, A.; Sakaguchi, S. Regulatory T Cells in Cancer Immunotherapy. Cell Res. 2017, 27, 109-118; Ménétrier-Caux, C.; Curiel, T.; Faget, J.; Manuel, M.; Caux, C.; Zou, W. Targeting Regulatory T Cells. Target. Oncol. 2012, 7, 15-28; Revilla SA et al., Colorectal Cancer-Infiltrating Regulatory T-Cells, Frontiers in Immunology 2022, 13: https://doi.org/10.3389/fimmu.2022.903564).

Based on their pivotal role of Tregs in suppressing the antitumor immune response, blocking their function and/or depleting them has emerged as a viable strategy to enhance or stimulate antitumor immunity (Dees et al., Eur. J. Immunol. 2021. 51:280-291). A variety of other approaches are currently under development including Treg cell depletion, suppressing their activity, impeding their recruitment, and preventing their differentiation within the TME.

The major challenge at present is how to specifically target Treg cells in the tumor site without affecting Tregs in blood circulation. It is important to avoid depleting the Tregs in the blood circulation because they serve the crucial function of maintaining immune homeostasis to prevent self-destruction of body tissues by the immune system. Depletion or inhibition of the Tregs in blood circulation would result in unwanted immune-related side effects such as autoimmunity or inflammation (Revilla SA et al., Colorectal Cancer-Infiltrating Regulatory T-Cells, Frontiers in Immunology 2022, 13: https://doi.org/10.3389/fimmu.2022.903564). Indeed, current treatments that deplete or interfere with the function of Tregs in the blood circulation also cause autoimmunity and other immune-related toxicities. To date, specific therapeutic approaches that specifically target Tregs within the TME without affecting Tregs in blood circulation are unavailable.

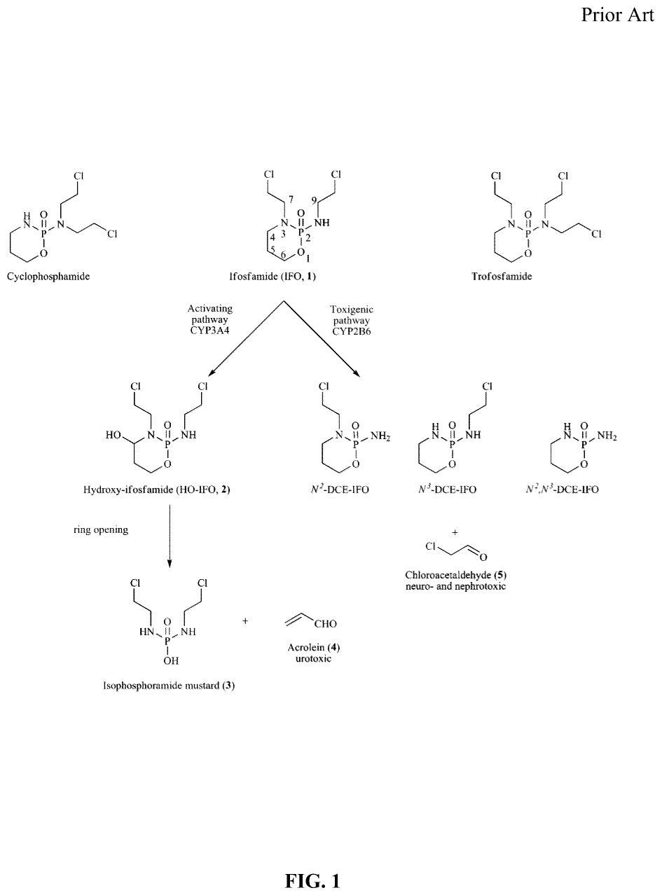

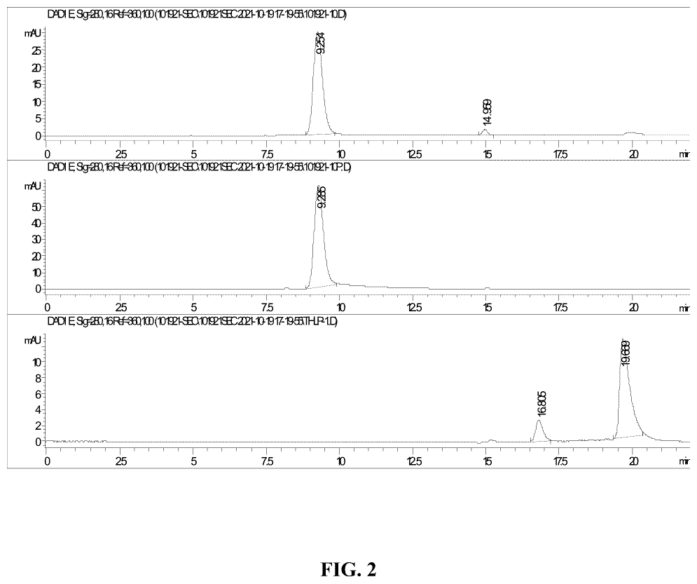

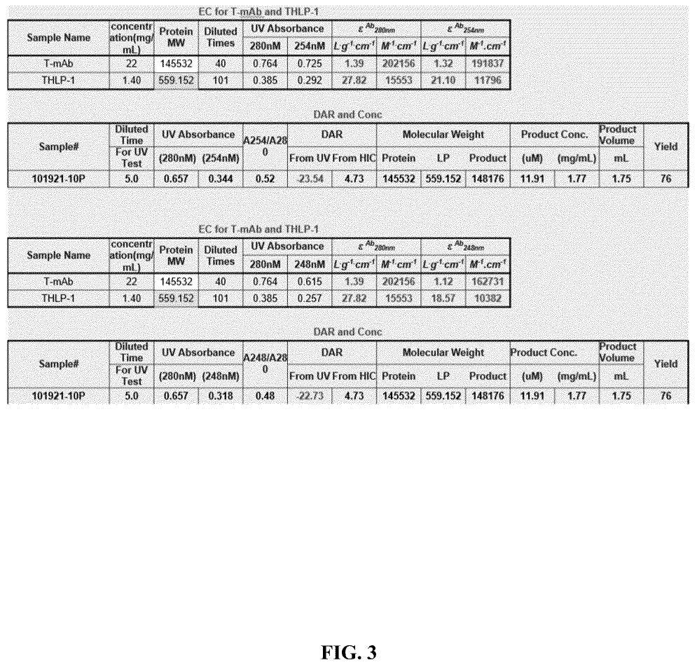

Examples of commonly used oxazaphosphorines are cyclophosphamide, ifosfamide and trofosfamide. Trofosfamide is mainly metabolized to ifosfamide and to a smaller extent to cyclophosphamide. Cyclophosphamide and ifosfamide are prodrugs that must be metabolized by the liver cytochrome p450 enzyme pathway to produce active cytotoxic metabolites (FIG. 1). They undergo activation through 4-hydroxylation followed by spontaneous degradation to the ultimate active and inactive metabolites. The activation is primarily mediated by CYP2B6, resulting in the formation of 4-hydroxycyclophosphamide, which is in equilibrium with aldophosphamide and aldoifosfamide the acyclic tautomer (Jeswani G and Paul S D, Recent Advances in the Delivery of Chemotherapeutic Agents. In Nano- and Microscale Drug Delivery Systems, 2017). The metabolites are then transported into the blood circulation and distribute throughout the body before reaching the target cancer cells. Phosphoramide mustard and isophosphoramide mustard are the final active antitumor metabolites of cyclophosphamide and ifosfamide, respectively. Other byproducts of metabolism of oxazaphosphorines are acrolein and chloroacetaldehydes, which cause the known toxic effects (bladder hemorrhage, neurotoxicity and urotoxicity of their precursors and other dose-limiting toxicities including bone marrow suppression, cardiotoxicity, diarrhea, and fatigue). Phosphoramide mustard and isophosphoramide mustard do not cause urotoxicity or neurotoxicity because they do not contain acrolein or chloroacetaldehydes. Derivatives or analogs of oxazaphosphorines that do not require bioactivation by liver CYP enzymes have also been synthesized. They include mafosfamide, glufosfamide and palifosfamide, evofosfamide, geranyloxy-isofosfamide or their analogs or derivatives. Low doses of oxazaphosphorines have significant immunomodulatory activities, notably the ability to suppress or deplete regulatory T cells (Tregs) and thereby counteract immunosuppression in the tumor microenvironment without affecting cytotoxic T cells or natural killer (NK) cells.

The approach of delivering oxazaphosphorines directly into the tumor microenvironment using ADCs can mitigate or significantly reduce the systemic toxicities.

The local delivery of the antibody moiety of the ADC also contributes to the antitumor effects of the ADC. The optimal effector function of the antibody moiety of the ADC can be improved by engineering the Fc region to increase its cytotoxic effects on cancer cells. Modifications can be made to improve affinity/avidity, antibody-dependent cellular cytotoxicity (ADCC), antibody-dependent cellular phagocytosis (ADCP) and complement-dependent cytotoxicity (CDC) by engineering the Fc region. Glyco-engineering such as afucosylation and amino acid mutation can be used. Pharmacokinetic profiles can also be increased by modifying the antibody to bind to neonatal Fc receptor, FcRn.

A rationale for using an oxazaphosphorine as payload in ADC is based on documented synergy of oxazaphosphorines with antibodies, immune checkpoint inhibitors or vaccines after systemic administration. However, toxicities associated with the combinations limit their use For example, survival synergy has been demonstrated when cyclophosphamide is combined with mAbs such as rituximab (anti-CD20 mAb), trastuzumab (anti-HER2 mAb) cetuximab (anti-EGFR mAb) or alemtuzumab (anti-CD52 mAb) (Roghanian A et al 2019, Cancer Immunol Res 7:1876-90; Nickenig C et al 2006, Cancer 107:1014-22; Scott D W 2014, Nat Rev Cancer 14:517-34; Keating M J et al 2005, J Clin Oncol 23:4079-88). Cyclophosphamide is also widely used as polychemotherapy (as part of combination with other chemotherapeutic drugs for treating breast cancer, leukemia, multiple myeloma, ovarian cancer, lymphoma, and others). Despite the synergy benefits, these combinations are fraught with dose-limiting toxicities and inconvenience. The combination treatment is time-consuming and inconvenient because each drug must be infused separately over prolonged duration (2-4 hours each) with rest periods between the infusions. Such an approach can also reduce patient compliance to treatment. The convenience of using an ADC is that it combines two drugs in one infusion and it minimizes systemic toxicity while maximizing efficacy. The ADCs can also be used in combination with other standard of care therapies including chemotherapy, immunotherapy, radiotherapy, phototherapy, gene therapy, cell therapy, oncolytic virus therapy or vaccination.

The disclosed invention comprises an antibody, linker, and payload. In an embodiment, the ADC increases accumulation of oxazaphosphorine payload within the tumor microenvironment and reduces systemic exposure and toxicities. In an embodiment, the payloads cause selective depletion of immunosuppressive cells such as Tregs within the tumor microenvironment; activate NK and effector T cells, reverse T cell exhaustion, change the functionality of immune cells within the tumor microenvironment to target cancer cells more effectively. The oxazaphosphorine payload also induces immunogenic cell death (ICD) of cancer cells and produces tumor-specific antigens that trigger long-lasting tumor-specific immunological memory.

Among advantages of the disclosure are indications for solid tumors and hematological malignancies, using combinations of therapies with clinically proven efficacy in cancer patients, novel immunomodulatory payloads and linkers, improved therapeutic index to eliminate or reduce systemic toxicities, convenience of administration (one drug instead of two separate administrations, and decreased development timelines from discovery to clinical trials.

The method can be used as a monotherapy or to complement standard of care chemotherapeutics, radiation, immunotherapy (antibodies including immune checkpoint inhibitors), cell therapy, gene therapy oncolytic virus therapy or vaccines.

“Immunotherapy” is used herein to refer to the administration of antibodies, antigen-binding fragments, antibody-drug conjugates (ADCs), and the like that bind to one or more proteins in the subject's body, and thereby affect a change in the protein's function that ameliorates the subject's cancer.

Radiation therapy may involve focusing a beam of radiation on the cancer site. Common radiation therapy regimens include sessions every weekday for 6-12 weeks.

Any of these therapies or a combination thereof may be chosen by the clinician, based on one or more aspects of the subject's presentation of the cancer, other aspects of the subject's health, one or more demographic factors that may be medically relevant, or one or more genetic markers of the subject, among other parameters.

In an embodiment, the immunostimulatory antibody-conjugates that target proteins on cancer cells such as HER2, EGFR, CD20, and TROP2 can be used to treat cancers solid tumors or hematological malignancies including, but not limited to, colorectal, breast, lung, gastric, lymphoma, leukemia, sarcomas, or head and neck, urothelial, renal, brain, uterine, ovarian, thyroid, melanoma, liver, pancreatic, HNSCC.

In an embodiment, the immunostimulatory ADCs include, but are not limited to, monospecific, bispecific, trispecific, or tetraspecific monoclonal antibodies.

Various methods can be used to synthesize antibody-drug conjugates. Yao et al., Int J Mol Sci. 2016 February; 17 (2): 194.

The subject to be treated can be a mammal such as a human, dog, cat, horse, or any other animal for which cancer treatment is desired.

In an embodiment, a drug can be attached to an antibody or antibody fragment utilizing a linker. In an embodiment, the linker is cleavable or non-cleavable. In an embodiment, the linker can be cleaved by various methods including proteolysis, reduction, or hydrolysis, hypoxia, or low pH.

In an embodiment, RTX5007, RTX5014, and RTX5015 is the linker payload.

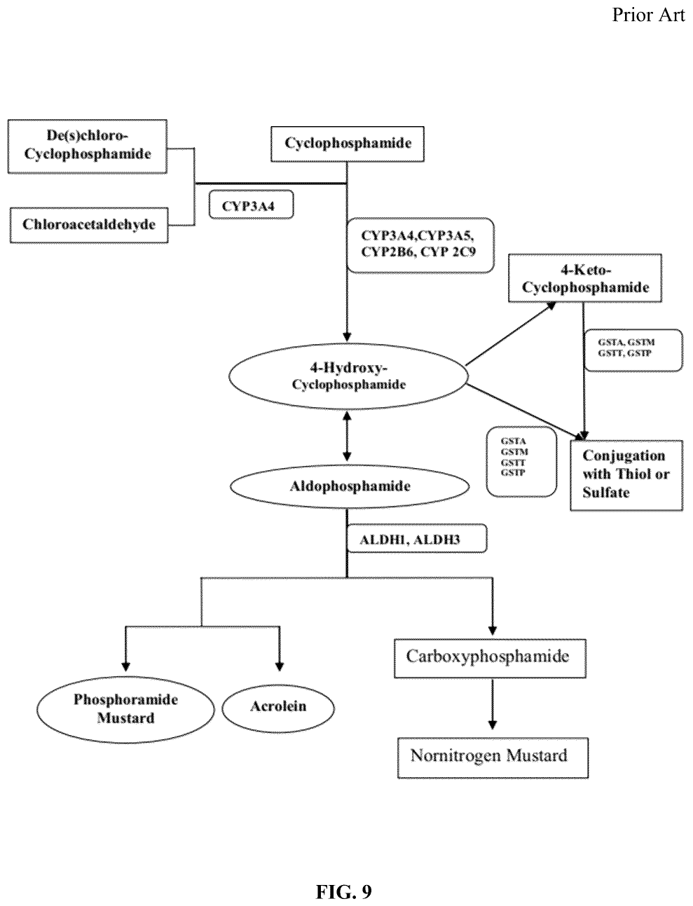

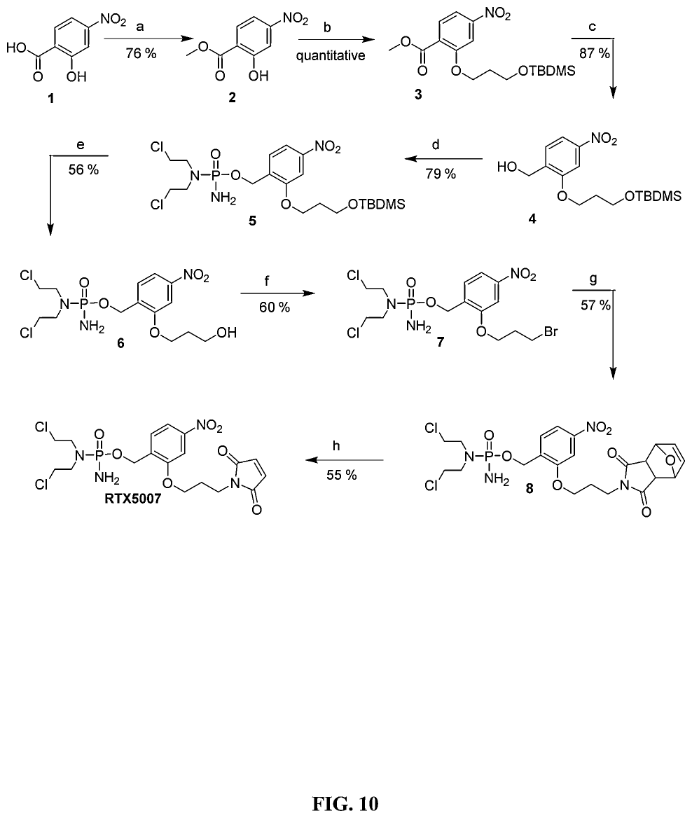

In an embodiment, the synthesis of RTX5007 was completed using the chemistry illustrated in FIG. 10 and Example 2.

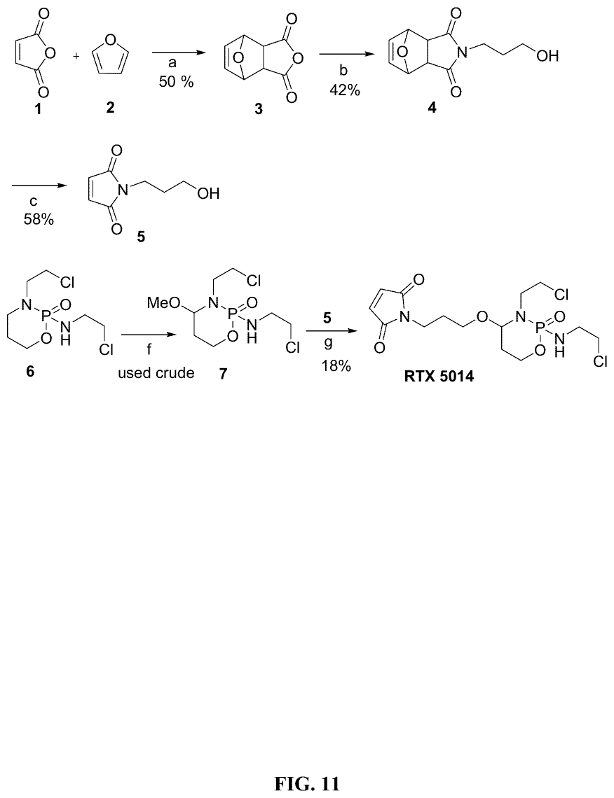

In an embodiment, the synthesis of RTX5014 was completed using the chemistry illustrated in FIG. 11 and Example 3.

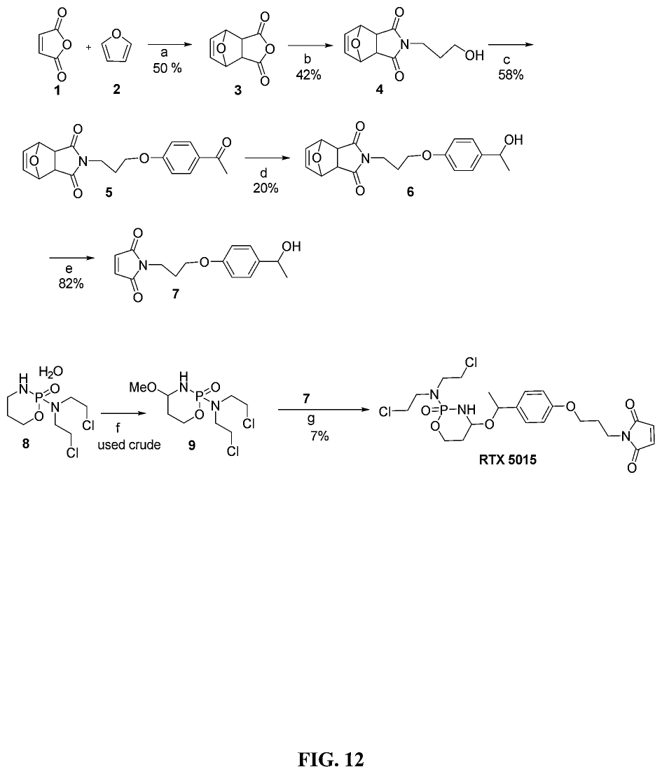

In an embodiment, the synthesis of RTX5015 was completed using the chemistry illustrated in FIG. 12 and Example 4.

In an embodiment, the antibody-drug conjugate Herceptin-RTX-5007. In an embodiment, Herceptin-RTX-5007 was made as in Example 5.

The RTX-5007 conjugation was scaled up to a 7.4 mg reaction with Herceptin at 4.9 mg/mL.

Herceptin was reduced using 6 eq of 10 mM TCEP, 29.4 L, at 37° C. for 1.5 h. After reduction 1 mM of EDTA was added to the protein from a 250 mM stock, 6.1 μL.

Prior to the addition of payload, the buffer composition was brought to 10% DMA with a 150 μL addition of DMA. After addition of the organic solvent, 15 eq of RTX-5007 was added to the mAb, 73.5 μL of 10 mM RTX-5007 in DMA. The mAb solution was mixed for 1.5 h at room temperature before DAR was checked. After confirmation of targeted DAR, 30 eq of 10 mM N-Acetyl Cysteine, 147 μL, was added to reaction for quenching RTX-5007.

Following conjugation, dialysis was preformed to remove excess payload using 1xPBS pH 7.4. After 3 exchanges of buffer over the course of 12 hours, the final buffer was changed to 1xPBS pH 7.4.

Following the removal of payload, 6.4 mg of ADC was recovered, and DAR was determined to be 7.8 by LC-MS. The monomeric purity was determined at 100% by SEC. The concentration was 4.0 mg/mL by A280.

FIG. 13 depicts monomer determination Herceptin-RTX-5007 by SEC (20 μg of conjugated injected over TSKgelG3000SWXL Mobile Phase 10% IPA in 1× PBS).

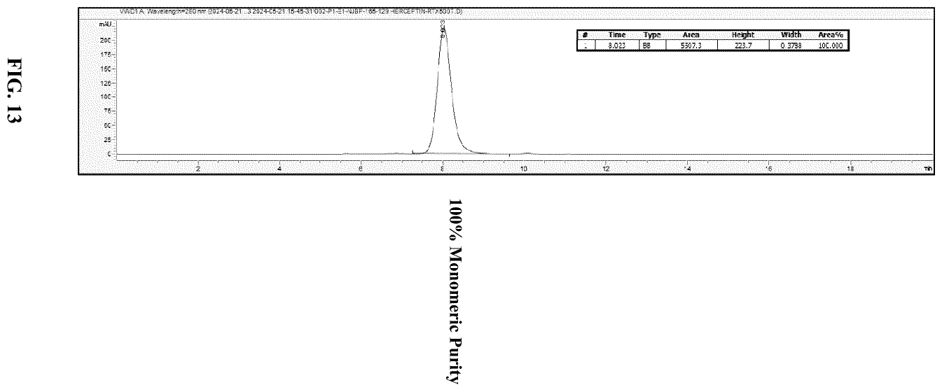

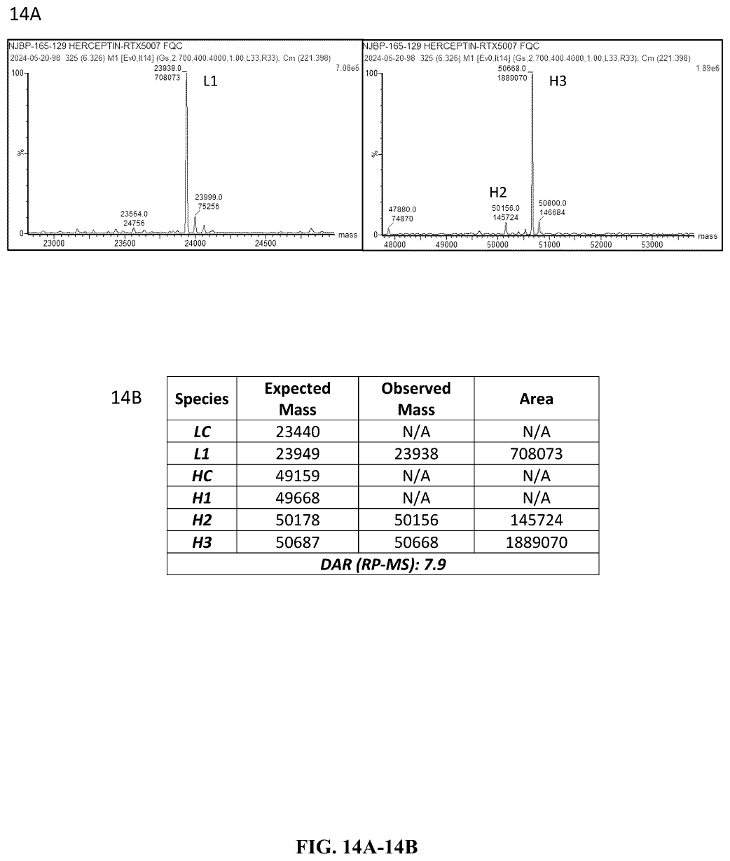

FIG. 14A-14B depict the drug-to-antibody ratio (DAR) determination Herceptin-RTX-5007 by LC-MS (0.2 μg of reduced material injected on PLRP-S(1000 Å, 8 μm 2.1 mm×50 mm)).

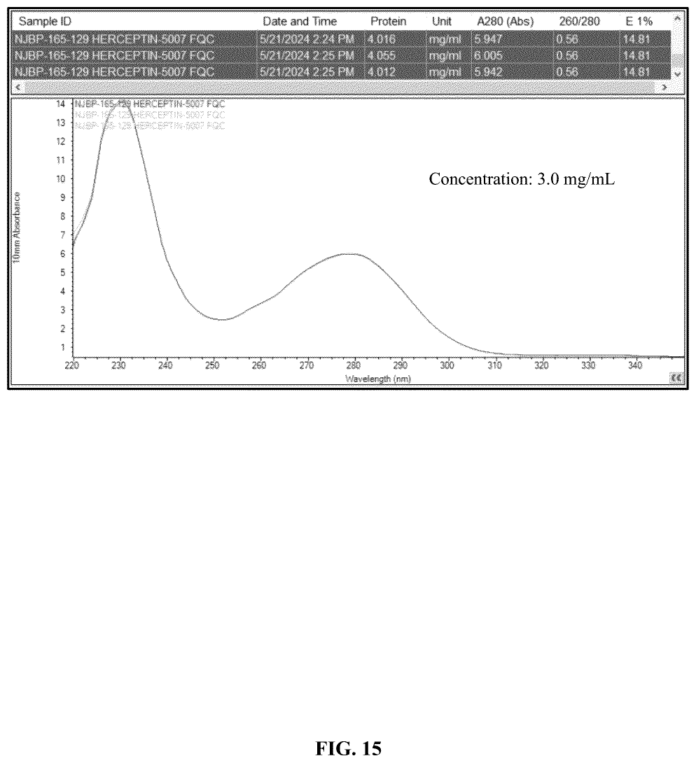

FIG. 15 depicts determination of the concentration of Herceptin-RTX-5007 of was determined by A280 on a nanodrop 2000C E1% with 14.8 extinction coefficient.

In an embodiment, the antibody-drug conjugate Sacituzumab-RTX-5007 Conjugate was made as in Example 6.

Prior to the conjugation, Sacituzumab was dialyzed into 1x PBS pH 7.4. The RTX-5007 conjugation was scaled up to a 10 mg reaction with Sacituzumab at 5.0 mg/mL.

Sacituzumab was reduced using 6 eq of 10 mM TCEP, 40 μL, at 37° C. for 1.5 h. After reduction 1 mM of EDTA was added to the protein from a 250 mM stock, 8.2 μL.

Prior to the addition of payload, the buffer composition was brought to 10% DMA with a 200 μL addition of DMA. After addition of the organic solvent, 20 eq of RTX-5007 was added to the mAb, 133.3 μL of 10 mM RTX-5007 in DMA. The mAb solution was mixed for 1.5 h at room temperature before DAR was checked. After confirmation of targeted DAR, 40 eq of 10 mM N-Acetyl Cysteine, 266.6 μL, was added to reaction for quenching RTX-5007.

Following conjugation, dialysis was preformed to remove excess payload using 1xPBS pH 7.4. After 3 exchanges of buffer over the course of 12 hours, the final buffer was changed to 1xPBS pH 7.4.

Following the removal of payload, 5.4 mg of ADC was recovered, and DAR was determined to be 8.0 by LC-MS. The monomer showed 100% by SEC. The concentration was 3.0 mg/mL by A280.

FIG. 16 depicts monomer determination of Sacituzumab-RTX-5007 by SEC (20 μg of conjugated injected over TSKgelG3000SWXL Mobile Phase 10% IPA in 1× PBS).

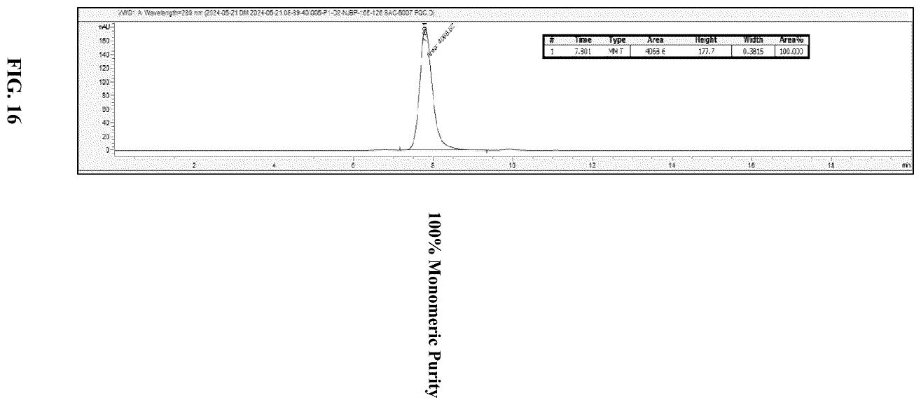

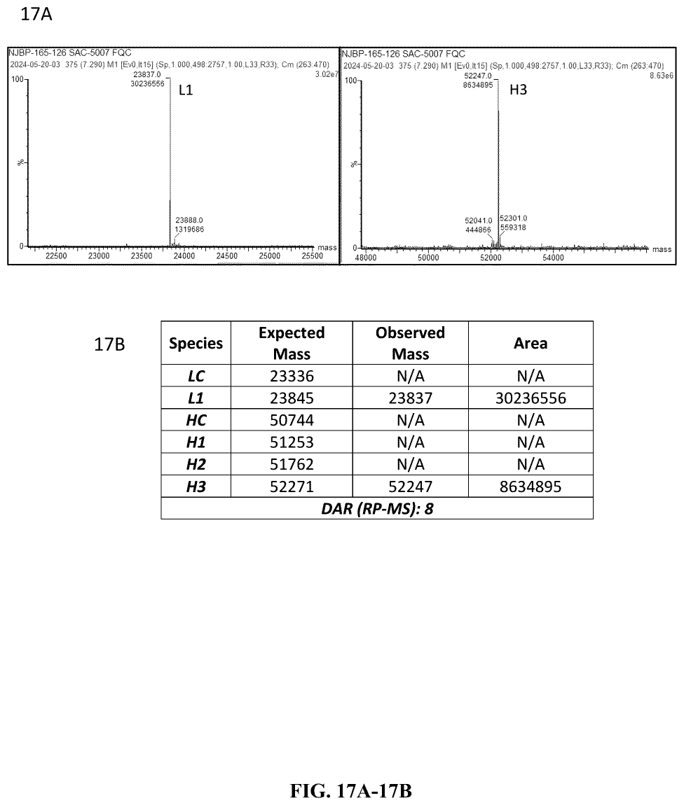

FIG. 17A-17B depict DAR determination of Sacituzumab-RTX-5007 by LC-MS (0.2 μg of reduced material injected on PLRP-S(1000 Å, 8 μm 2.1 mm×50 mm)).

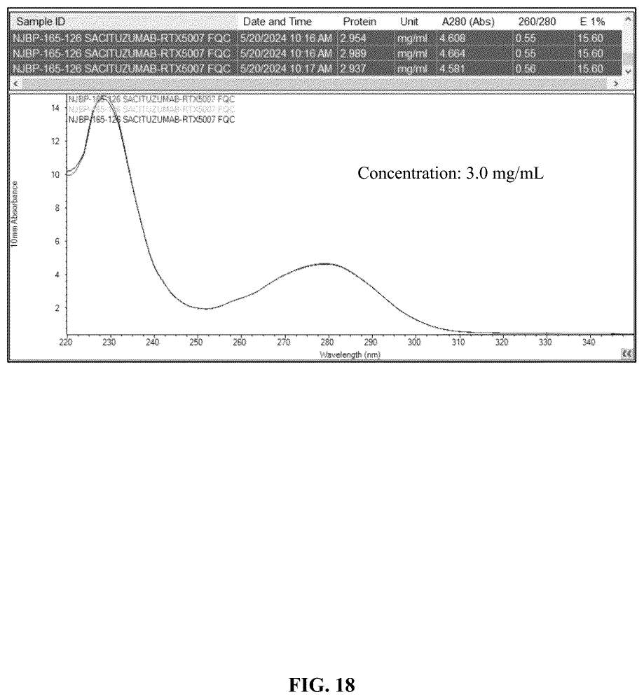

FIG. 18 depicts determination of the concentration of Sacituzumab-RTX-5007 was determined by A280 on a nanodrop 2000C E1% with 15.6 extinction coefficient.

In an embodiment, the binding kinetics of an antibody-drug conjugate using an anti-HER2 antibody (Herceptin) and an antibody-drug conjugate using an anti-TROP2 antibody (Sacituzumab) were determined.

Herceptin-ADC or Sacituzumab-ADC were used at various concentration to determine the interaction kinetics. Herceptin or Sacituzumab was used as positive control and human Isotype IgG were used as negative control at the same concentration dilutions.

His-tagged human HER2 or His-tagged human Trop2 (˜35 nM and 150 nM respectively) were loaded on Ni-NTA probe by dipping the probe for 180-300 seconds. The His-tagged receptor loaded probe was dipped into various concentrations (500 nM, 250 nM, 125 nM, 62.5 nM) of either Herceptin-ADC/Sacituzumab-ADC or mAbs for 300 seconds to determine association kinetics. The His-tagged receptor probe was dipped into Q-buffer for 300 seconds to determine dissociation kinetics of the bound ADCs/mAbs.

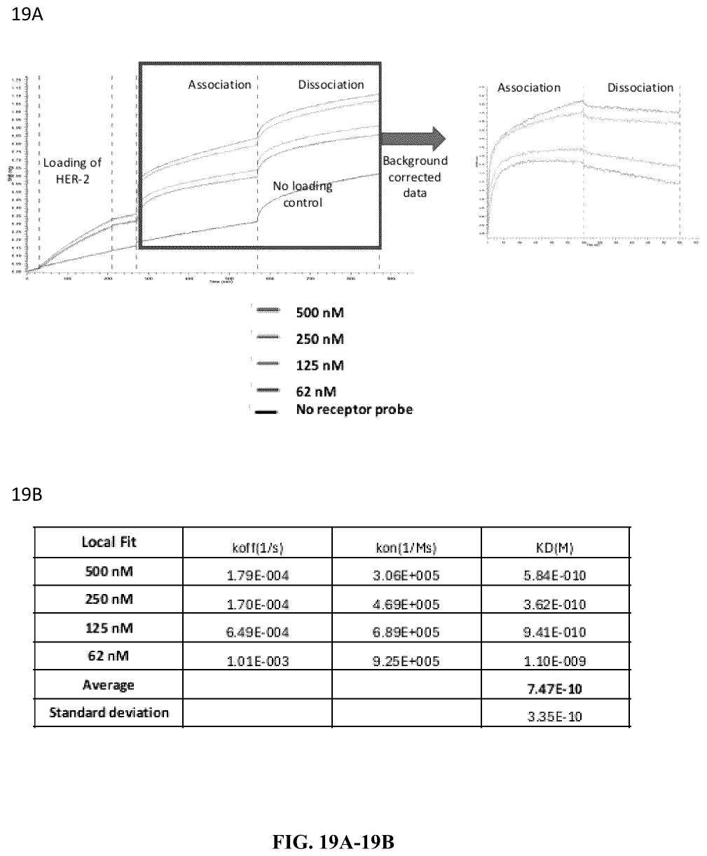

FIG. 19A-19B depict the binding kinetics of Herceptin to HER2.

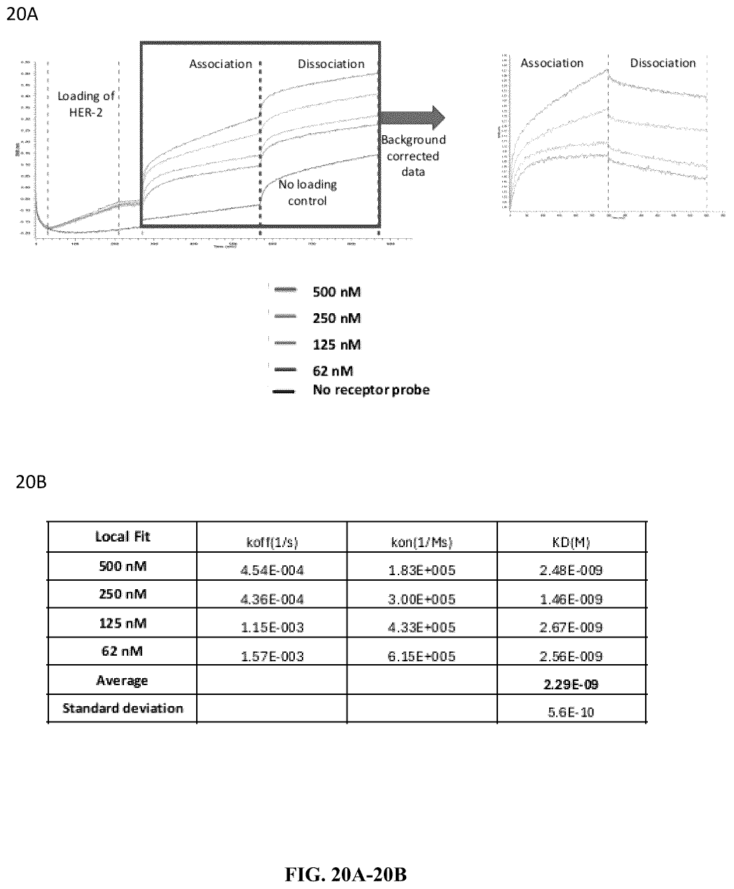

FIG. 20A-20B depict the binding kinetics of Herceptin-ADC to HER2.

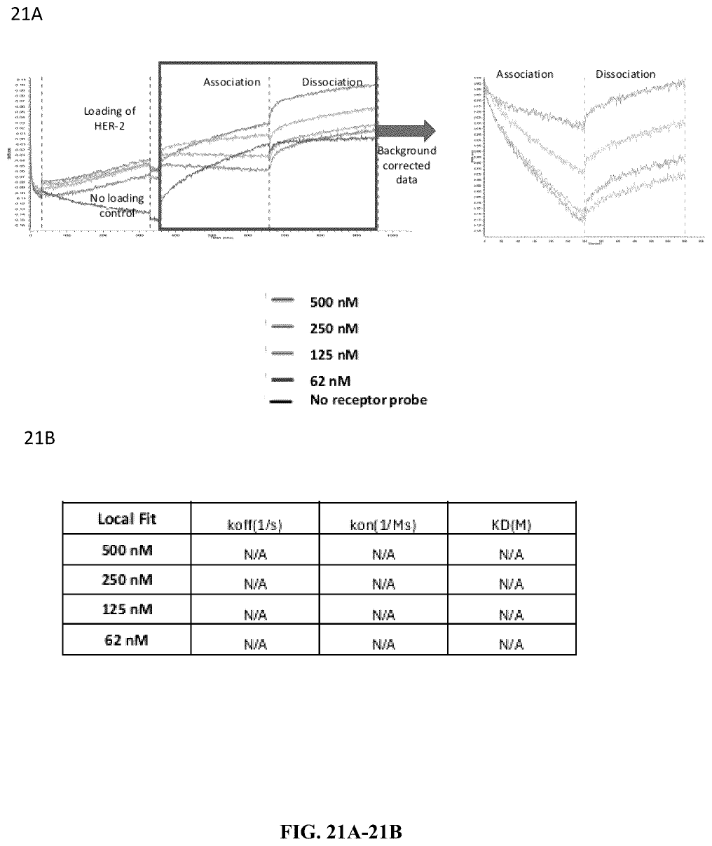

FIG. 21A-21B depict the binding kinetics of Isotype to HER2.

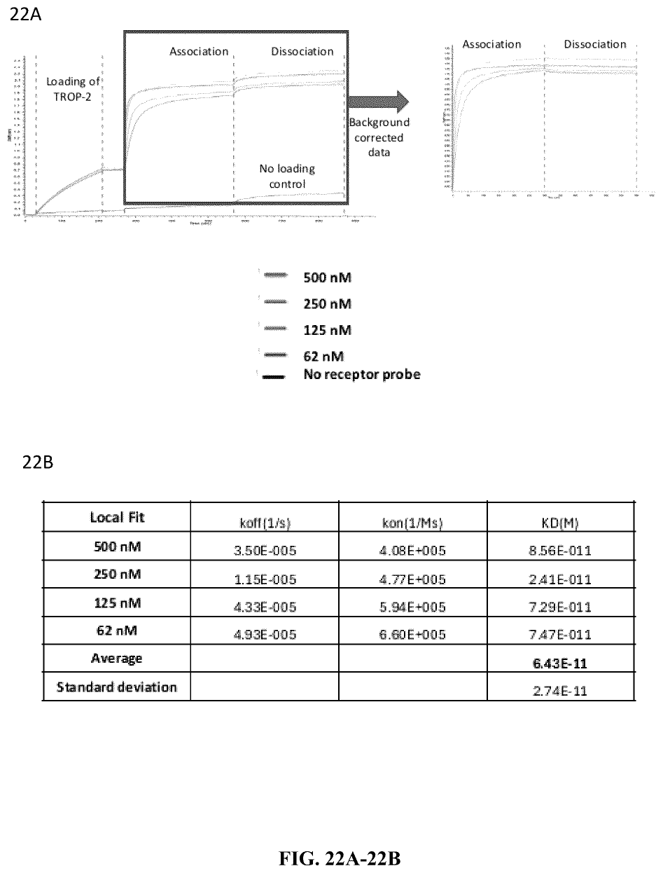

FIG. 22A-22B depict the binding kinetics of Sacituzumab to TROP2.

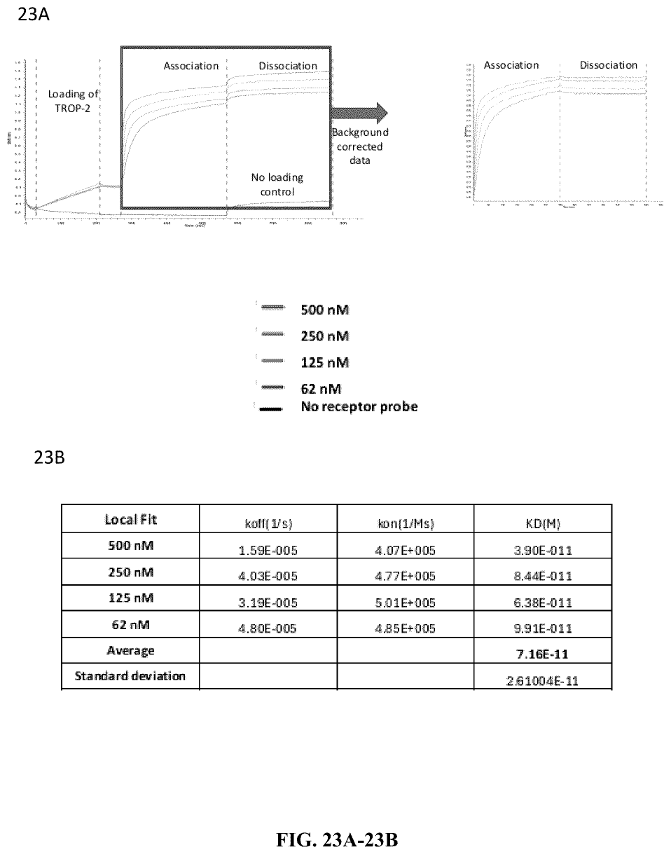

FIG. 23A-23B depict the binding kinetics of Sacituzumab-ADC to TROP2.

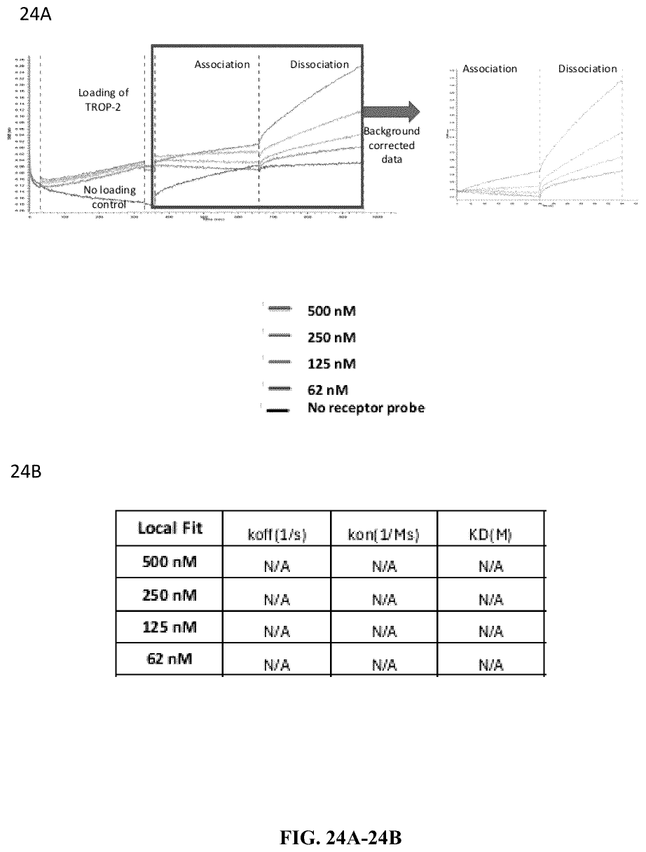

FIG. 24A-24B depict the binding kinetics of Isotype to TROP2.

Both Herceptin and its ADC derivative showed comparable binding to HER-2 receptor (about 0.5-2 nM range), indicating minimal effect of conjugation to the receptor binding ability of mAb. Similarly, Sacituzumab and its ADC showed comparable binding to TROP-2 receptor (about 0.04-0.1 nM range).

In an embodiment, in vitro data on cytotoxicity of antibody-drug conjugate using an anti-HER2 antibody (Herceptin) and antibody-drug conjugate using an anti-TROP2 antibody (Sacituzumab) was determined.

Herceptin-ADC is an antibody-drug conjugate (ADC) targeting Her2 which delivers a small molecule (oxazaphosphorine) for antitumor activity. N87 cell line is reported to express high levels of Her2 mRNA transcripts and protein.

Sacituzumab-ADC is an antibody-drug conjugate (ADC) targeting Trop2 which delivers a small molecule (oxazaphosphorine) payload for antitumor activity. N87 cell line is reported to express moderate levels of Trop2 mRNA transcripts.

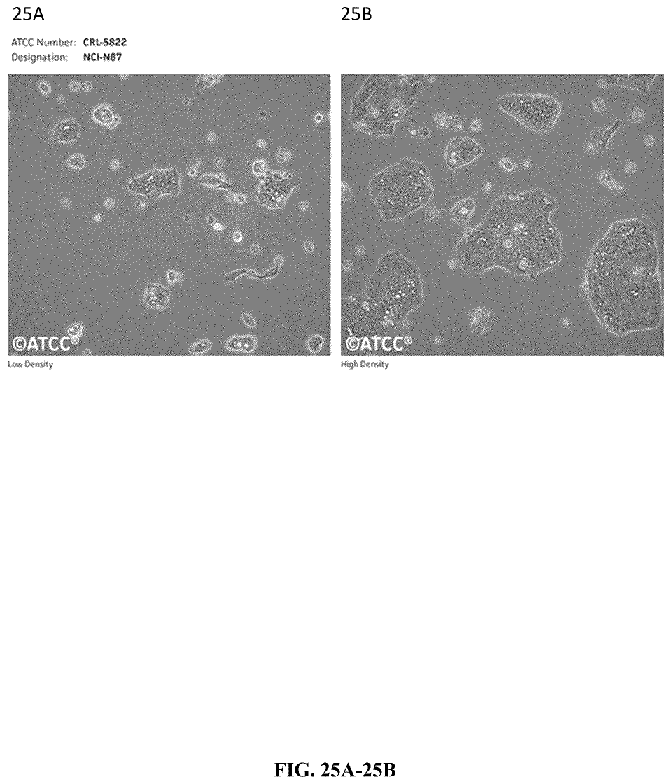

FIG. 25A-25B depicts live cell image analysis (IncuCyte S3) assessment of NCI-N87 cells across a range of seeding densities (FIG. 25A low density; FIG. 25B high density) in 96-well plates to monitor cell growth for up to 120 Hrs.

In an embodiment, the in vitro cytotoxicity study design was as follows. NCI-N87 cells were seeded in 96-well plates at 30,000 cells/well and allowed to adhere overnight in normal growth media (RPMI-10% FBS). N87 cells were treated with either Sacituzumab-ADC (NJBP-0165-126) or Herceptin-ADC (NJBP-0165-129) across dose levels ranging from 30 mg/mL to 0.03 mg/mL in the presence of Caspase 3/7 and imaged every six hours on phase contrast to monitor % confluence and 488 nm fluorescence to monitor apoptosis. IC50 curves were plotted for 24, 48, 72-74 and 96 hours of culture. In an embodiment, the cell density can vary depending on the cell line utilized.

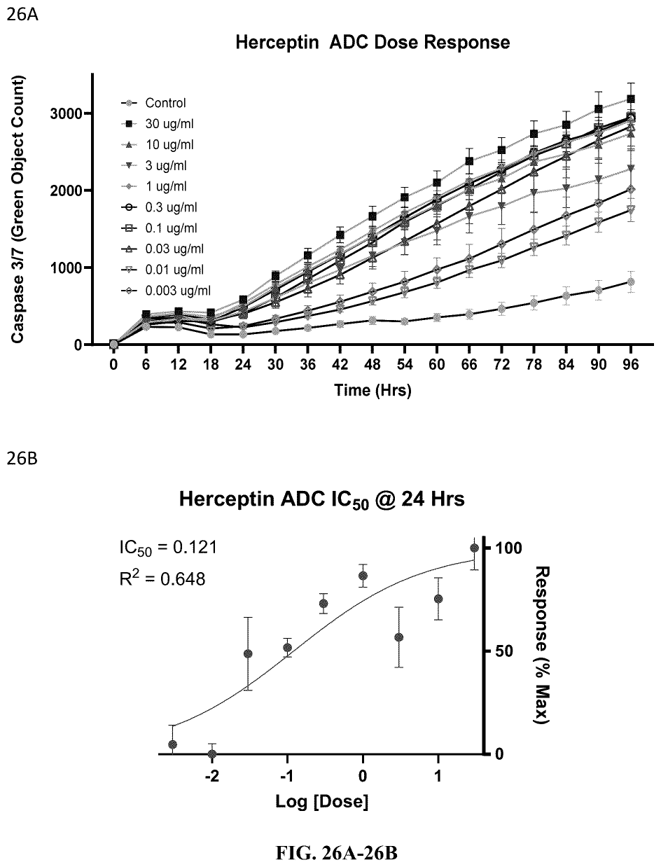

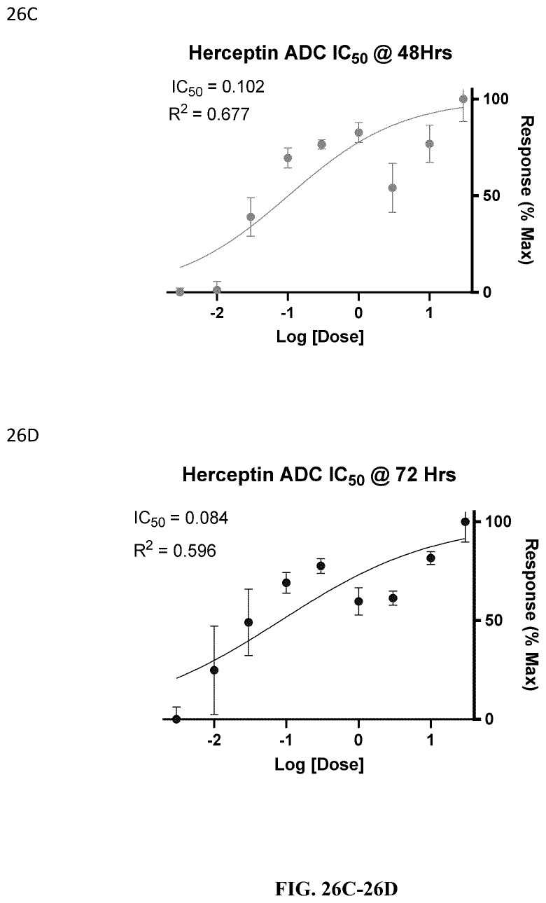

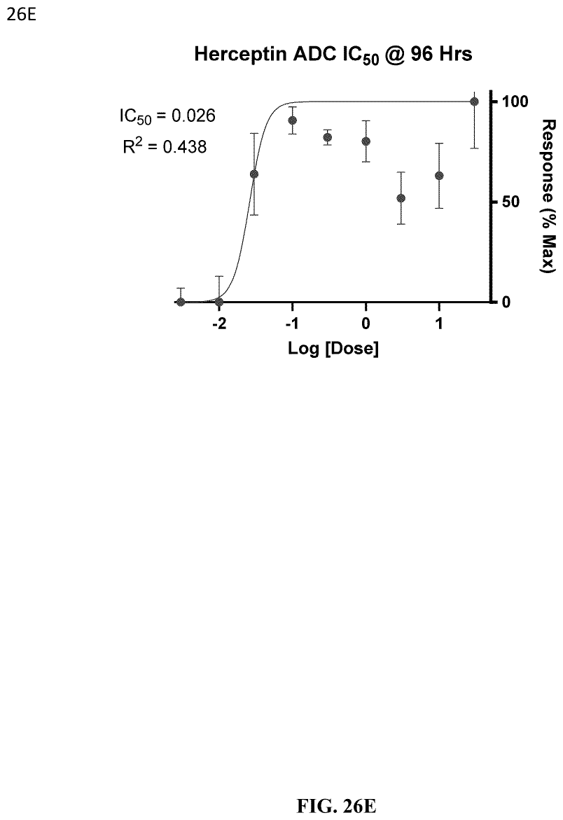

FIG. 26A depicts N87 cells treated with Herceptin-ADC (NJBP-0165-129) across dose levels ranging from 30 mg/mL to 0.03 mg/mL in the presence of Caspase 3/7 and imaged every six hours on phase contrast to monitor % confluence and 488 nm fluorescence to monitor apoptosis, FIG. 26B-26E depict IC50 curves plotted for 24 (26B), 48 (26C), 72 (26D), and 96 (26E) hours of culture.

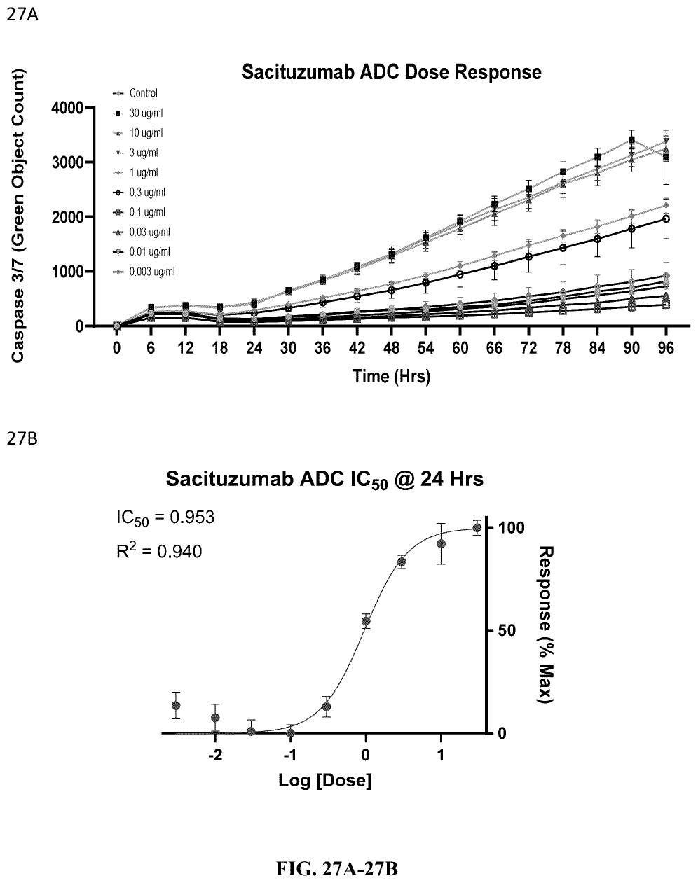

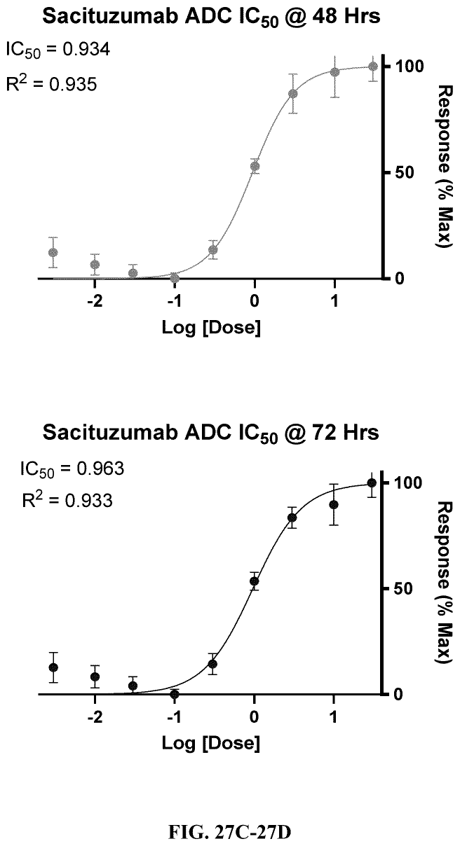

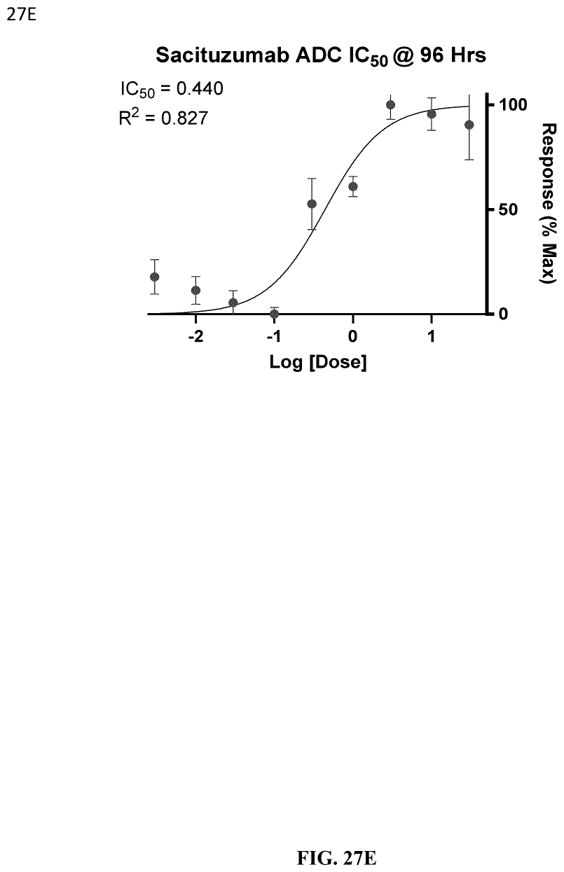

FIG. 27A depicts N87 cells treated with Sacituzumab-ADC (NJBP-0165-126) across dose levels ranging from 30 mg/mL to 0.03 mg/mL in the presence of Caspase 3/7 and imaged every six hours on phase contrast to monitor % confluence and 488 nm fluorescence to monitor apoptosis; FIG. 27B-27E depict IC50 curves plotted for 24 (27B), 48 (27C), 72 (27D), and 96 (27E) hours of culture.

The results show a dose and target dependent response to the ADC. The results show target (HER2 expression) and dose-dependent cytotoxicity to Herceptin-ADC and Sacituzumab-ADC in N87 cells. N87 cells are more sensitive to Herceptin-ADC (IC50=0.026 μg/mL) than Sacituzumab-ADC (IC50=0.440 μg/mL) at 96 hours.

In an embodiment, the antibody in the ADC is trastuzumab (Herceptin).

In an embodiment, the antibody in the ADC the antibody is sacituzumab.

In an embodiment, the active oxazaphosphorine payload in the ADC is 10-oxa-4-azatricyclo [5.2.1.0,2,6]dec-8-ene-3,5-dione.

In an embodiment, the linker and active oxazaphosphorine payload in the ADC are RTX5007.

In an embodiment, the antibody-drug conjugate is trastuzumab-RTX5007.

In an embodiment, the antibody-drug conjugate is sacituzumab-RTX5007.

In an embodiment, the method for treating cancer comprises administering an ADC wherein the antibody is trastuzumab.

In an embodiment, the method for treating cancer comprises administering an ADC wherein the antibody is sacituzumab.

In an embodiment, the method for treating cancer comprises administering an ADC wherein the active oxazaphosphorine payload is 10-oxa-4-azatricyclo [5.2.1.0,2,6]dec-8-ene-3,5-dione.

In an embodiment, the method for treating cancer comprises administering an ADC wherein the linker and active oxazaphosphorine payload is RTX5007.

In an embodiment, the method for treating cancer comprises administering an ADC wherein the antibody-drug conjugate is trastuzumab-RTX5007.

In an embodiment, the method for treating cancer comprises administering an ADC wherein the antibody-drug conjugate is sacituzumab-RTX5007.

The disclosure also relates to pharmaceutical compositions that can comprise an immunostimulatory ADC that can be administered to a subject for the purposes of including, but not limited to, treating cancer or other hyperproliferative diseases. Compositions comprising the ADC can be optimized and made suitable for administration to a subject via intravenous, subcutaneous, intramuscular, intradermal, or intracerebral routes. The pharmaceutical composition can be supplied as a liquid solution, a suspension, an emulsion, or as solid forms suitable for dissolution or suspension in liquid prior to use.

The pharmaceutical composition can comprise a pharmaceutically acceptable carrier, which is to say, any carrier that does not interfere with the effectiveness of the biological activity of the ingredients and that is not toxic to the subject to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

Suitable carriers and their formulations are described in Remington: The Science and Practice of Pharmacy, 21st Edition, David B. Troy, ed., Lippincott Williams & Wilkins (2005). Typically, an appropriate amount of a pharmaceutically acceptable salt is used in the formulation to render the formulation isotonic, although the formulate can be hypertonic or hypotonic if desired. Examples of the pharmaceutically acceptable carriers include, but are not limited to, sterile water, saline, buffered solutions like Ringer's solution, and dextrose solution. The pH of the solution is generally about 5 to about 8 or from about 7 to 7.5. Certain carriers may be more preferable depending upon, for instance, the route of administration and concentration of composition being administered. Carriers suitable for direct delivery may be administered without undue toxicity. Pharmaceutically acceptable excipients include, but are not limited to, sorbitol, any of the various tween compounds, and liquids such as water, saline, glycerol, and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Most preferably, the composition is combined with saline, Ringer's balanced salt solution (pH 7.4), and the like.

The pharmaceutical composition disclosed herein can be formulated for administration. The amount of pharmaceutical composition to be administered may be determined by standard procedure well known by those of ordinary skill in the art. Physiological data of the patient (e.g. age, size, and weight) and type and severity of the disease being treated have to be taken into account to determine the appropriate dosage.

The pharmaceutical composition may be formulated for administration by injection, e.g., by intravenous, intramuscular, subcutaneous, or intradermal injection. Formulations for injection may be presented in unit dosage form, e.g., in ampoules or in multi-dose containers. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Optionally, the pharmaceutical composition may be frozen for storage at any temperature appropriate for storage.

### Gene Therapy

Gene therapy is the modification of cells to insert a polynucleotide into cells, e.g., as an episome and/or integrated into the genome, wherein the polynucleotide provides a therapeutic effect, by encoding a polypeptide for which expression is desired, disrupting a genomic sequence encoding a polypeptide which is desired to be silenced, modulating the transcription of an endogenous sequence encoding a polypeptide, or the like. Generally, the modification may be brought about by preparing a vector comprising the polynucleotide and using the vector to insert the polynucleotide into the nuclei of one or more cells.

Suitable gene therapy vectors are well-known in the art and include, for example AAV vectors, and retroviral vectors (e.g. lentiviral vectors, gamma retroviral vectors).

### Kits

Also disclosed herein are kits comprising the ADC herein or a pharmaceutical composition thereof.

The kit may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), to reconstitute the complex to form a solution for injection purposes.

A kit can further comprise a needle or syringe, preferably packaged in sterile form, for injecting the complex, and/or a packaged alcohol pad. Instructions are optionally included for administration of compositions by a clinician or by the patient.

### Methods

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following examples, which are introduced for illustration only and not intended to be limiting.

### EXAMPLES

The following are examples of methods and compositions of the invention. It is understood that various other embodiments may be practiced, given the general description provided herein.

### Example 1. Preparation of Trastuzumab-THLP1as a Prototype Oxazaphosphorine-ADC Using a Hydrazone Linker

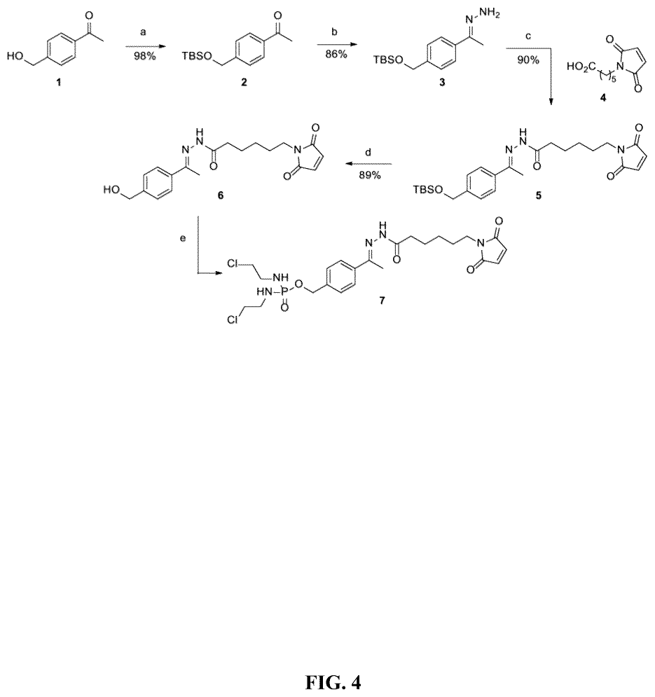

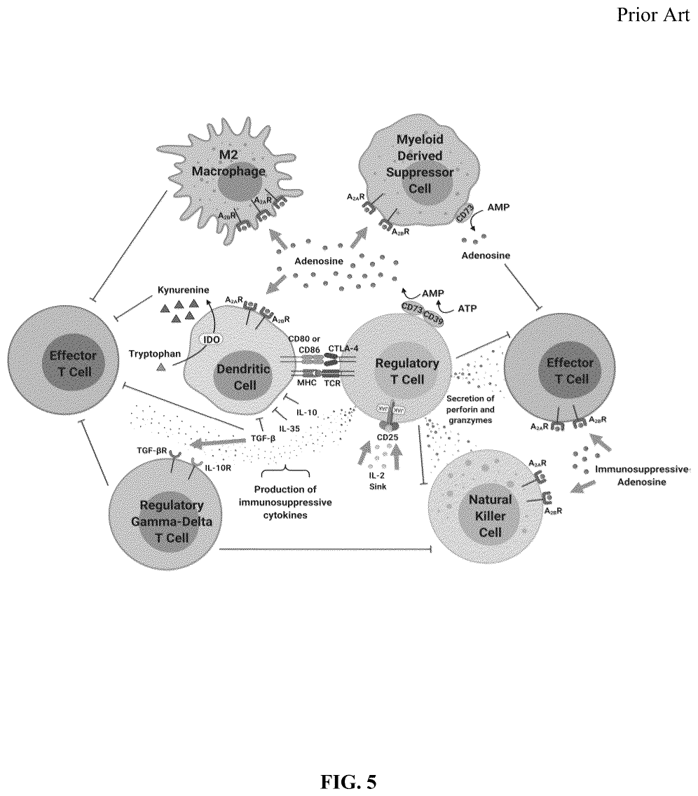

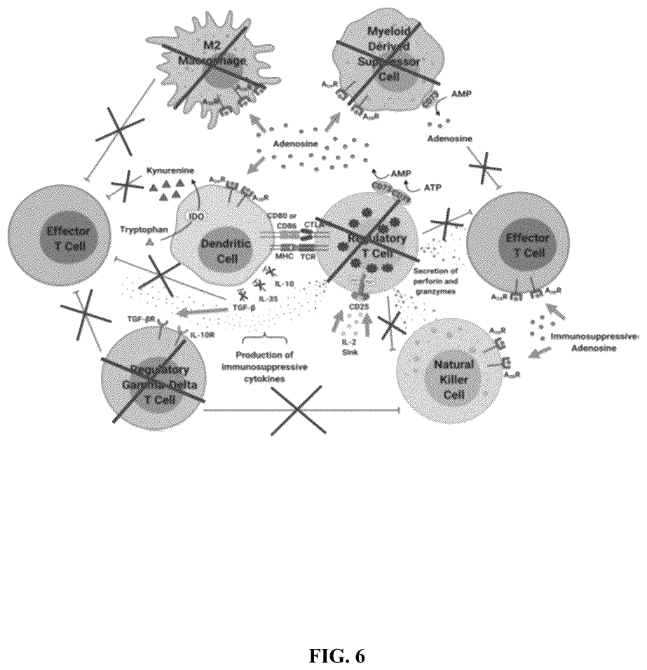

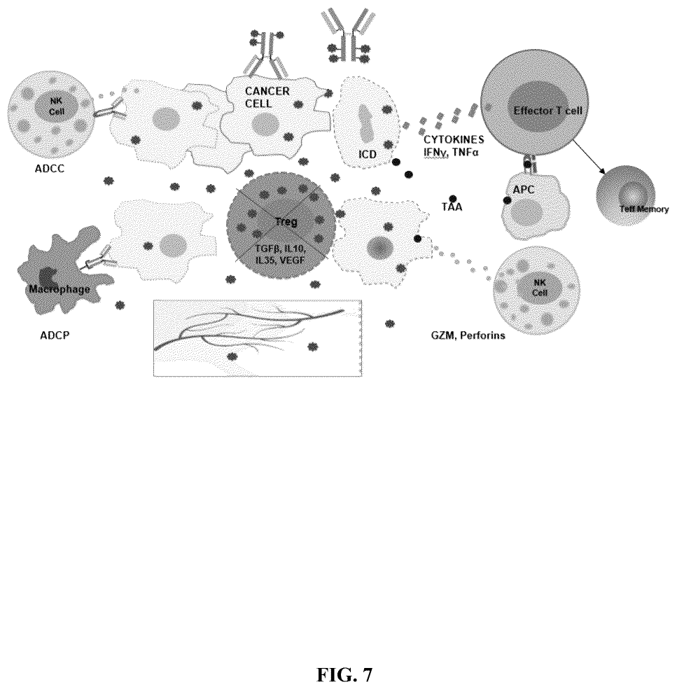

The preparation of Trastuzumab-THLP1 was completed in two parts: first the synthesis of the linker payload, and second, preparation of the corresponding ADC, Trastuzumab-THLP1. The synthesis of the linker payload was completed as depicted in FIG. 4.

Reagents and reaction conditions: (a) tert-butyldimethylsilyl chloride, imidazole, DMF; (b) hydrazine hydrate, acetic acid, ethanol; (c) oxalyl chloride, triethylamine, DMF; (d) TBAF, acetic acid, THF; (e) POCI3, 2-chloroethylamine hydrochloride, triethylamine, DCM.

Treatment of 1-[4-(hydroxymethyl)phenyl]ethan-1-one (1) with tert-butyldimethylsilyl chloride and imidazole in DMF gave protected alcohol 2 in 98% yield. The protected alcohol was treated with hydrazine hydrate and acetic acid in ethanol to give desired hydrazone 3 in 86% yield. Activation of 6-(2,5-dioxo-2,5-dihydro-1H-pyrrol-1-yl) hexanoic acid with oxalyl chloride in dichloromethane using catalytic DMF gave the corresponding acid chloride which was coupled with hydrazone 3 to give acylhydrazone 5. Deprotection of the tert-butyldimethylsilyl group using TBAF buffered with acetic acid gave alcohol 6. Finally, treatment with POCI3 at −78° C. in dichloromethane followed by 2-chloroethylamine hydrochloride and triethylamine gave the target compound 7.

Using the linker payload, the target ADC was prepared using the following procedure: trastuzumab solution (800.0 μL, 5 mg/mL) was added with TCEP solution in PBS buffer (156.7 μL, 0.5 mM, pH=7), the resulted solution was swirled gently and incubated at 33° C. for 1 hour; Linker payload THLP-1 (66.0 μL, 2.5 mM in DMSO) was added, the solution was swirled gently and kept at 25° C. for 0.5 hour. The crude sample was purified by Illustra NAP-10 Columns (GE Healthcare) and filtered through 0.25 μm microfilter to afford the final ADC TmAb-THLP1, 1.75 mL, 1.77 mg/mL, DAR=4.73, UmAb %=3.33%, Agg %=0.80%.

Other linkers to conjugate an antibody to a payload are well-known to those skilled in the art {Yao H et al. Int. J. Mol. Sci. (2016), 17, 194; doi: 10.3390/ijms17020194; Doronina S O et al. Nat. Biotechnol. 21 (2003) 778-784; Hamann P R et al. Chem. 13 (2002) 47-58; Backer B S et al. Tetrahedron Lett. (2020) 61 (12): doi: 10.1016/j.tetlet.2; Tang H et al. Front. Pharmacol. (2019) 10:373. doi: 10.3389/fphar.2019.00373}; Khongoruzul P et al. Mol Cancer Res 2020; 18:3-19 doi: 10.1158/1541-7786.MCR-19-0582; Baah S et al Molecules 2021, 26, 2943. https://doi. org/10.3390/molecules26102943

### Example 2. Synthesis of Linker-Payload RTX5007 (Phosphoramide Mustard)

RTX5007 synthesis was completed in 8 steps from commercially available 2-hydroxy-4-nitrobenzoic acid 1. The synthesis of RTX5007 was completed using the chemistry illustrated in FIG. 10.

Reagents and reaction conditions: (a) methanol, sulphuric acid; (b) (3-bromopropoxy)-tert-butyldimethylsilane, potassium carbonate, DMF; (c)diisobutylaluminium hydride, THF; (d) lithium bis(trimethylsilyl)amide, bis(2-chloroethyl)phosphoramidic dichloride, ammonia, THF; (e) hydrochloric acid, water, methanol; (f) carbon tetrabromide, triphenylphosphine, dichloromethane; (g) 10-oxa-4-azatricyclo [5.2.1.0,2,6]dec-8-ene-3,5-dione, potassium carbonate, DMF; (h) toluene. Treatment of 2-hydroxy-4-nitrobenzoic acid 1 with sulphuric acid in methanol gave ester 2. Purification by silica gel column gave 76% yield; confirmed by 1H NMR (>95% NMR purity). The ester 2 was treated with (3-bromopropoxy)-tert-butyldimethylsilane in the presence of potassium carbonate in DMF gave the protected ester 3. Purification by silica gel column gave quantitative yield; confirmed by 1H NMR (>95% NMR purity). The protected ester 3 was reduced using diisobutylaluminium hydride (DIBAL-H) in THF afforded alcohol 4. Purification by silica gel column gave 87% yield; confirmed by 1H NMR (>95% NMR purity). Treatment of alcohol 4 with lithium bis(trimethylsilyl)amide at −78 0C followed by bis(2-chloroethyl)phosphoramidic dichloride then ammonia in THF gave phosphoramide 5. Purification by silica gel column gave 79% yield; confirmed by 1H NMR (>95% NMR purity). Phosphoramide 5 was deprotected with aqueous hydrochloric acid (1M) in methanol to afford alcohol 6. Purification by silica gel column gave 56% yield; confirmed by 1H NMR (>95% NMR purity). Alcohol 6 was treated with carbon tetrabromide and triphenylphosphine in dichloromethane to give bromide 7. Purification by silica gel column gave 60% yield; confirmed by 1H NMR and MS ES+=492, 494 (>95% NMR purity). Displacement of bromide 7 with 10-oxa-4-azatricyclo [5.2.1.0,2,6]dec-8-ene-3,5-dione in the presence of potassium carbonate in DMF afforded protected phthalimide 8. Purification by silica gel column gave 57% yield; confirmed by 1H NMR and MS ES+=577 (>95% NMR purity).

Finally, reverse Diels-Alder of compound 8 in toluene at 120° C. gave the target compound RTX5007.

Crude RTX5007 was purified by silica gel column gave 55% yield. The product was confirmed by 1H NMR, 31P NMR, MS ES+=509 and HPLC (Purity=98.1%). 100 mg was ready for biological testing.

### Example 3. Synthesis of Linker-Payload RTX5014 (Isophosphoramide Mustard)

Synthesis of RTX5014 was completed in 5 steps from commercially available Maleic anhydride 1 and furan 2. The synthesis of RTX5014 was completed using the chemistry illustrated in FIG. 11.

Reagents & Conditions: a) Diethyl ether, rt. 24 h; b) 3-amino-1-propanol MeOH,0OC-rt, o/n; c) toluene, 130 00C, 6h; f) MeOH, anodic oxidation; g) BF3·OEt2, DCM.

Procedure: Reaction of maleic anhydride (25 g, 253.5 mmol, 1 eq.) with furan (34.5 g, 507 mmol, 1 eq.) in diethyl ether (250 ml) at room temperature for overnight provided the bicyclic Diels-alder adduct 3 (21 g) in 50% yield as white powder which was filtered and washed with cold diethyl ether. The crude material was sufficiently pure by NMR to be used in next step without further purification. Reaction of 3-amino-1-propanol (4.5 g, 60 mmol, 1 eq.) with the 3 (10 g, 60 mmol, 1 eq.) in MeOH provided 4 (5.6 g) as white solid in 42% yield which was used as such in next step without further purification. The refluxing of 4 (3 g, 13.44 mmol) in toluene (10 ml) at 1300c for 6 hr provided the required compound 5 (1.6 g) in 75% yield as a waxy solid after silica gel chromatographic purification (Structure confirmed by 1H NMR, purity 95%).

Commercially available Ifosfamide 6 (300 mg, 1.1 mmol, 1 eq) was subjected to electrochemical anodic oxidation (J. Med. Chem. 2015, 58, 705-717) to provide 7 (300 mg, 96%, crude yield), which was used immediately without any further purification owing to high instability. The formation of 7 was confirmed by TLC (DCM: acetone 1:1). The reaction of 7 (300 mg, 1.1 mmol, 1 eg.) with 5 (356 mg, 2.3 mmol, 2 eq.) in presence of BF3·OEt (152 mg, 1.1 mmol, 1 eq.) at −78° C. in DCM provided crude RTX5014 (523 mg). Mass analysis of the crude reaction mixture confirmed the product formation. Crude reaction mixture was purified by silica gel column chromatography. 1H NMR of the isolated compound showed desired product with minor impurities, yield (87 mg, 18%, Purity ˜85% by NMR). The NMR of the sample recorded after 4 days showed further decomposition. The bulk stored sample was repurified to remove the impurities to provide 27 mg of RTX5014 (Purity ˜85% by NMR).

### Example 4. Synthesis of Linker-Payload RTX5015

Synthesis of RTX5015 was completed in 7 steps from commercially available Maleic anhydride 1 and furan 2. The synthesis of RTX5015 was completed using the chemistry illustrated in FIG. 12.

Reagents & Conditions: a) Diethyl ether, rt. 24 h; b) 3-amino-1-propanol MeOH,0OC-rt, o/n; c) PPh3, DIAD, 4-hydroxyacetophenone, 0° C.-rt, o/n; d) DCM: MeOH (3:1), NaBH4, rt, 4h; e) toluene, 130 00C, 6h; f) MeOH, anodic oxidation; g) BF3.OEt2, DCM.

Procedure: Reaction of maleic anhydride (25 g, 253.5 mmol, 1 eq.) with furan (34.5 g, 507 mmol, 1 eq.) in diethyl ether (250 ml) at room temperature for overnight provided the bicyclic Diels-alder adduct 3 (21 g) in 50% yield as white powder which was filtered and washed with cold diethyl ether. The crude material was sufficiently pure by NMR to be used in next step without further purification. Reaction of 3-amino-1-propanol (4.5 g, 60 mmol, 1 eq.) with the 3 (10 g, 60 mmol, 1 eq.) in MeOH provided 4 (5.6 g) as white solid in 42% yield which was used as such in next step without further purification. Reaction of 4 (6 g, 27 mmol, 1 eq.) with 4-hydroxyacetopheone (3.7 g, 27 mmol eq.), DIAD (6 g, 30 mmol, 1.1 eq.), PPh3 (7.7 g, 30 mmol, 1 eq.) in THF under Mitsunobu conditions provided 5 (5.2 g) in 58% yield after column chromatographic purification (Structure confirmed by 1H NMR, purity 95%). Reduction of 5 (5 g, 14.6 mmol, 1 eq.) with NaBH4 (1.1 g, 29.3 mmol, 2 eq.) in DCM: MeOH (3:1) mixture provided the desired alcohol 6 (1 g) in 20% yield after column chromatographic purification (Structure confirmed by 1H NMR). The refluxing of 6 (175 mg, 0.5 mmol) in toluene (3 ml) at 1300c for 6 hr provided the required compound 7 (115 mg) in 82% yield as a waxy solid after silica gel chromatographic purification (Structure confirmed by 1H NMR, purity 95%).

Commercial cyclophosphamide 8 (300 mg, 1.1 mmol, 1 eq) was subjected to electrochemical anodic oxidation (J. Med. Chem. 2015, 58, 705-717) to provide 9 (300 mg, 96%, crude yield), which was used immediately without any further purification as 9 has been reported to be very unstable. The formation of 9 was confirmed by TLC (DCM: acetone 1:1). The reaction of 9 (300 mg, 1 mmol, 1 eg.) with 7 (443 mg, 16 mmol, 1.5 eq.) in presence of BF3.OEt (152 mg, 1 mmol, 1 eq.) at −78° C. in DCM provided crude RTX5015 (533 mg). Mass analysis of the crude reaction mixture confirmed the product formation. Crude reaction mixture was purified by silica gel column chromatography. 1H NMR of the isolated compound showed desired product with multiple stereoisomeric product, Yield (38 mg, 7%, Purity ˜85% by NMR). However, the presence of multiple product stereoisomers made it very difficult to calculate their exact ratios and overall purity. The presence of the characteristic product signals in 1H NMR along with the mass spectrometric data supports the formation of the product and overall purity. Product stability was checked after 48h by NMR and was found to be stable at −20° C.

### Example 5. Making of Antibody-Drug Conjugate Using an Anti-HER2 Antibody (Trastuzumab (Herceptin))

### Herceptin-RTX-5007

The RTX-5007 conjugation was scaled up to a 7.4 mg reaction with Herceptin at 4.9 mg/mL.

Herceptin was reduced using 6 eq of 10 mM TCEP, 29.4 μL, at 37° C. for 1.5 h. After reduction 1 mM of EDTA was added to the protein from a 250 mM stock, 6.1 μL.

Prior to the addition of payload, the buffer composition was brought to 10% DMA with a 150 μL addition of DMA. After addition of the organic solvent, 15 eq of RTX-5007 was added to the mAb, 73.5 μL of 10 mM RTX-5007 in DMA. The mAb solution was mixed for 1.5 h at room temperature before DAR was checked. After confirmation of targeted DAR, 30 eq of 10 mM N-Acetyl Cysteine, 147 μL, was added to reaction for quenching RTX-5007.

Following conjugation, dialysis was preformed to remove excess payload using 1xPBS pH 7.4. After 3 exchanges of buffer over the course of 12 hours, the final buffer was changed to 1xPBS pH 7.4.

Following the removal of payload, 6.4 mg of ADC was recovered, and DAR was determined to be 7.8 by LC-MS. The monomeric purity was determined at 100% by SEC. The concentration was 4.0 mg/mL by A280.

FIG. 13 depicts monomer determination Herceptin-RTX-5007 by SEC (20 μg of conjugated injected over TSKgelG3000SWXL Mobile Phase 10% IPA in 1× PBS).

FIG. 14A-14B depict the drug-to-antibody ratio (AR) determination Herceptin-RTX-5007 by LC-MS (0.2 μg of reduced material injected on PLRP-S(1000 Å, 8 μm 2.1 mm×50 mm)).

FIG. 15 depicts determination of the concentration of Herceptin-RTX-5007 of was determined by A280 on a nanodrop 2000C E1% with 14.8 extinction coefficient.

### Example 6. Making of Antibody-Drug Conjugate Using an Anti-TROP2 Antibody (Sacituzumab). Sacituzumab-RTX-5007 Conjugate

Prior to the conjugation, Sacituzumab was dialyzed into 1x PBS pH 7.4. The RTX-5007 conjugation was scaled up to a 10 mg reaction with Sacituzumab at 5.0 mg/mL.

Sacituzumab was reduced using 6 eq of 10 mM TCEP, 40 μL, at 37° C. for 1.5 h. After reduction 1 mM of EDTA was added to the protein from a 250 mM stock, 8.2 μL.

Prior to the addition of payload, the buffer composition was brought to 10% DMA with a 200 μL addition of DMA. After addition of the organic solvent, 20 eq of RTX-5007 was added to the mAb, 133.3 μL of 10 mM RTX-5007 in DMA. The mAb solution was mixed for 1.5 h at room temperature before DAR was checked. After confirmation of targeted DAR, 40 eq of 10 mM N-Acetyl Cysteine, 266.6 μL, was added to reaction for quenching RTX-5007.

Following conjugation, dialysis was preformed to remove excess payload using 1×PBS pH 7.4. After 3 exchanges of buffer over the course of 12 hours, the final buffer was changed to 1xPBS pH 7.4.

Following the removal of payload, 5.4 mg of ADC was recovered, and DAR was determined to be 8.0 by LC-MS. The monomer showed 100% by SEC. The concentration was 3.0 mg/mL by A280.

FIG. 16 depicts monomer determination of Sacituzumab-RTX-5007 by SEC (20 μg of conjugated injected over TSKgelG3000SWXL Mobile Phase 10% IPA in 1× PBS).

FIG. 17A-17B depict DAR determination of Sacituzumab-RTX-5007 by LC-MS (0.2 μg of reduced material injected on PLRP-S(1000 Å, 8 μm 2.1 mm×50 mm)).

FIG. 18 depicts determination of the concentration of Sacituzumab-RTX-5007 was determined by A280 on a nanodrop 2000C E1% with 15.6 extinction coefficient.

### Example 7. Binding Kinetics of Antibody-Drug Conjugate Using an Anti-HER2 Antibody (Herceptin) and an Antibody-Drug Conjugate Using an Anti-TROP2 Antibody (Sacituzumab)

Probe Used: Ni-NTA; Buffer: Q buffer, K buffer (Q-buffer: 0.2% BSA, 0.02% Tween-20, 0.05% Proclin 300 in PBS; K-buffer 10% Q-buffer diluted in PBS) All the receptors and mAbs were diluted in K buffer and Q-buffer was used to initiate dissociation of the complex.

Loading concentration of His-tagged human HER2 (2.5 μg/mL) or His-tagged human Trop2 (5 μg/mL) (receptor for ADCs) to Ni-NTA probe.

Herceptin-ADC/Sacituzumab-ADC was used at various concentration to determine the interaction kinetics. Herceptin/Sacituzumab is used as positive control and human Isotype IgG were used as negative control at the same concentration dilutions.

His-tagged human HER2 or His-tagged human Trop2 (˜35 nM and 150 nM respectively) was loaded on Ni-NTA probe by dipping the probe for 180-300 seconds; additional probe was used for background corrections that was not loaded with His-tagged receptor but used ADCs/mAbs in association step.

His-tagged receptor loaded probe was dipped into various concentrations (500 nM, 250 nM, 125 nM, 62.5 nM) of either Herceptin-ADC/Sacituzumab-ADC or mAbs for 300 seconds to determine association kinetics.

His-tagged receptor probe was dipped into Q-buffer for 300 seconds to determine dissociation kinetics of the bound ADCs/mAbs.

The data is presented as individual concentration dependent calculations (Local fit).

FIG. 19A-19B depict the binding kinetics of Herceptin to HER2. His-tagged receptor loaded probe was dipped into various concentrations (500 nM, 250 nM, 125 nM, 62.5 nM) of either Herceptin-ADC or mAbs for 300 seconds to determine association kinetics. FIG. 19B depicts the individual concentration dependent calculations (Local fit).

FIG. 20A-20B depict the binding kinetics of Herceptin-ADC to HER2. His-tagged receptor loaded probe was dipped into various concentrations (500 nM, 250 nM, 125 nM, 62.5 nM) of Herceptin-ADC for 300 seconds to determine association kinetics. FIG. 20B depicts the individual concentration dependent calculations (Local fit).

FIG. 21A-21B depict the binding kinetics of Isotype to HER2. His-tagged receptor loaded probe was dipped into various concentrations (500 nM, 250 nM, 125 nM, 62.5 nM) of Herceptin-ADC for 300 seconds to determine association kinetics. FIG. 21B depicts the individual concentration dependent calculations (Local fit).

FIG. 22A-22B depict the binding kinetics of Sacituzumab to TROP2. His-tagged receptor loaded probe was dipped into various concentrations (500 nM, 250 nM, 125 nM, 62.5 nM) of Sacituzumab-ADC for 300 seconds to determine association kinetics. FIG. 22B depicts the individual concentration dependent calculations (Local fit).

FIG. 23A-23B depict the binding kinetics of Sacituzumab-ADC to TROP2. His-tagged receptor loaded probe was dipped into various concentrations (500 nM, 250 nM, 125 nM, 62.5 nM) of Sacituzumab-ADC for 300 seconds to determine association kinetics. FIG. 23B depicts the individual concentration dependent calculations (Local fit).

FIG. 24A-24B depict the binding kinetics of Isotype to TROP2. His-tagged receptor loaded probe was dipped into various concentrations (500 nM, 250 nM, 125 nM, 62.5 nM) of mAbs for 300 seconds to determine association kinetics. FIG. 24B depicts the individual concentration dependent calculations (Local fit).

Both Herceptin and ADC derivative showed comparable binding to HER-2 receptor (about 0.5-2 nM range), indicating minimal effect of conjugation to the receptor binding ability of mAb.

Similarly, Sacituzumab and its ADC showed comparable binding to TROP-2 receptor (about 0.04-0.1 nM range).

The human Isotype IgG (negative control) did not bind to either of the receptors.

### Example 8. In Vitro Data on Cytotoxicity of Antibody-Drug Conjugate Using an Anti-HER2 Antibody (Herceptin) and Antibody-Drug Conjugate Using an Anti-TROP2 Antibody (Sacituzumab)

Herceptin-ADC is an antibody-drug conjugate (ADC) targeting Her2 which delivers a small molecule (oxazaphosphorine) for antitumor activity.

The N87 cell line is reported to express high levels of Her2 mRNA transcripts (https://www.proteinatlas.org/ENSG00000141736-ERBB2/cell+line) and protein (Cancer Cell International. 2014: https://cancerci.biomedcentral.com/articles/10.1186/1475-2867-14-10).

Sacituzumab-ADC is an antibody-drug conjugate (ADC) targeting Trop2 which delivers a small molecule (oxazaphosphorine) payload for antitumor activity.

The N87 cell line is reported to express moderate levels of Trop2 mRNA transcripts (https://www.proteinatlas.org/ENSG00000184292-TACSTD2/cell+line).

FIG. 25A-25B depict live cell image analysis (IncuCyte S3) assessment of NCI-N87 cells across a range of seeding densities (FIG. 25A low density; FIG. 25B high density) in 96-well plates to monitor cell growth for up to 120 Hrs.

In an embodiment, the in vitro cytotoxicity study design was as follows. The NCI-N87 cell line was purchased from ATCC and expanded in culture prior to use in in vitro assays. Cells were first assessed across a range of seeding densities in 96-well plates to monitor cell growth for up to 120 Hrs using Live cell image analysis (IncuCyte S3). Seeding density of 30,000 cells per well was selected for dose response studies. NCI-N87 cells were seeded in 96-well plates at 30,000 cells/well and allowed to adhere overnight in normal growth media (RPMI-10% FBS). N87 cells were treated with either Sacituzumab-ADC (NJBP-0165-126) or Herceptin-ADC (NJBP-0165-129) across dose levels ranging from 30 mg/mL to 0.03 mg/mL in the presence of Caspase 3/7 (CellEvent™ Green Reagent, Invitrogen) and imaged every six hours on phase contrast to monitor % confluence and 488 nm fluorescence to monitor apoptosis. IC50 curves were plotted for 24, 48, 72-74 and 96 hours of culture.

FIG. 26A depicts N87 cells treated with Herceptin-ADC (NJBP-0165-129) across dose levels ranging from 30 mg/mL to 0.03 mg/mL in the presence of Caspase 3/7 (CellEvent™ Green Reagent, Invitrogen) and imaged every six hours on phase contrast to monitor % confluence and 488 nm fluorescence to monitor apoptosis, FIG. 26B-26E depict IC50 curves plotted for 24 (26B), 48 (26C), 72 (26D), and 96 (26E) hours of culture.

FIG. 27A depicts N87 cells treated with Sacituzumab-ADC (NJBP-0165-126) across dose levels ranging from 30 mg/mL to 0.03 mg/mL in the presence of Caspase 3/7 (CellEvent™ Green Reagent, Invitrogen) and imaged every six hours on phase contrast to monitor % confluence and 488 nm fluorescence to monitor apoptosis; FIG. 27B-27E depict IC50 curves plotted for 24 (27B), 48 (27C), 72 (27D), and 96 (27E) hours of culture.

The results show a dose and target dependent response to the ADC. The results show target (HER2 expression) and dose-dependent cytotoxicity to Herceptin-ADC and Sacituzumab-ADC in N87 cells. N87 cells are more sensitive to Herceptin-ADC (IC50=0.026 μg/mL) than Sacituzumab-ADC (IC50=0.440 μg/mL) at 96 hours.

We claim:

1. A composition comprising an antibody-drug conjugate of the formula Ab-(L-D)ₙ wherein Ab is an antibody, antibody fragment, antibody chain, affibody, aptamer, or a nanobody;
D is an active oxazaphosphorine payload;
L is a linker; and
n has a value of 2 to 20.

2. The composition of claim 1, wherein n has a value of 2-8.

3. The composition of claim 1 wherein the antibody is trastuzumab.

4. The composition of claim 1 wherein the antibody is sacituzumab.

5. The composition of claim 1 wherein the linker and active oxazaphosphorine payload is RTX5007.

6. The composition of claim 1 wherein the antibody-drug conjugate is trastuzumab-RTX5007.

7. The composition of claim 1 wherein the antibody-drug conjugate is sacituzumab-RTX5007.

8. The composition of claim 1, wherein the Ab binds to a tumor-associated antigen from at least one of a group comprising HER2, HER3, VEGF-A, VEGFR-2, CSF-1R, PD-L1, CEACAM5 or CEACAM6, ROR1, CD20, CD19, CD22, CD30, CD33, CD133, CD38, CD39 CD25, CD47, CD52, CD56, CD70, CD73, CD74, CD79b, CD155, CD166, FGF-receptor, B7-H3, B7-H4, LIV1, PSMA, PSCA, MAGE-A4, EpCAM, IL1R, CCR8, CCR4, Claudin, APPL2, BCMA, EGFR, DLL3/4, SSX-2, Tissue Factor, folate receptor, mesothelin receptor, NaPi2b, 5T4, Nectin-4, Nectin-2 (CD112), c-MeT, Trop-2, LHRH (GnRH) receptor, gonadotropin (LH/hCG, FSH) receptor, prolactin receptor, claudins; survivin, STEAP1, Transferrin receptor 1, NRG1, EphB2, and Caveolin-1.

9. The composition of claim 1, wherein the oxazaphosphorine payload is cytotoxic to immunosuppressive T-regulatory cells.

10. The composition of claim 1, wherein the oxazaphosphorine payload is cytotoxic to a cancer cell.

11. The composition of claim 1, wherein the oxazaphosphorine payload is of the formula: 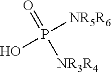
wherein at least one of R₃, R₄, R₅, and R₆ is a CH₂CH₂Y;
wherein Y is a halogen; and
wherein the remaining R₃, R₄, R₅, and R₆ groups are a hydrogen or a lower alkyl group.

12. The composition of claim 11, wherein the halogen is Cl or Br.

13. The composition of claim 11, wherein the hydrogen in R₃ and R₅ are replaced with deuterium to form CD₂CH₂ Y or methyl (CH₃) to form CH₃CH₂Y.

14. The composition of claim 11, wherein the oxazaphosphorine payload is of a structure selected from the group consisting of 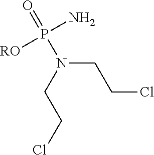
wherein R is an alkyl chain; 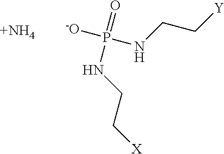
wherein X and Y are halogen leaving groups; 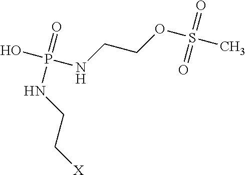
wherein X is a halogen leaving group; and 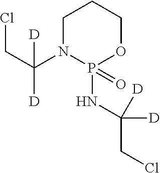
wherein D is deuterium.

15. The composition of claim 11, wherein the oxazaphosphorine payload is selected from 4-hydroxycyclophosphamide, aldophosphamide, phosphoramide mustard, 3-hydroxypropanal, isophosphoramide mustard, 4-hydroxycyclophosphamide, 4-hydroperoxycyclophosphamide, 4-hydroxyifosfamide, 4-hydroperoxyifosfamide evofosfamide, mafosfamide, glufosfamide, or trifosfamide mustard.

16. The composition of claim 11, wherein the oxazaphosphorine payload is selected from an analog or derivative of 4-hydroxycyclophosphamide, aldophosphamide, phosphoramide mustard, 3-hydroxypropanal, isophosphoramide mustard, 4-hydroxycyclophosphamide, 4-hydroperoxycyclophosphamide, 4-hydroxyifosfamide, 4-hydroperoxyifosfamide evofosfamide, mafosfamide, glufosfamide, or trifosfamide mustard.

17. The composition of claim 16 wherein the payload is phosphoramide mustard.

18. The composition of claim 16, wherein the derivative of 4-hydroperoxyifosfamide is 4-hydroxyifosfamide.

19. The composition of claim 16, wherein the analog or derivative of 4-hydroperoxyifosfamide (4-HO-ifosfamide) is deuterated (d4-hydroxyifosfamide).

20. The composition of claim 16, wherein the 4-hydroperoxycyclophosphamide derivative is 4-hydroxycyclophosphamide.

21. The composition of claim 11, wherein the oxazaphosphorine payload is of the structure: 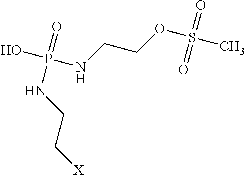
wherein X is Cl or Br.

22. The composition of claim 20, wherein X is Cl.

23. The composition of claim 11, wherein the oxazaphosphorine payload is of the structure: 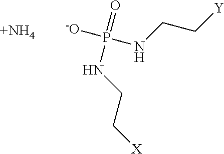
wherein X and Y represent independent leaving groups.

24. The composition of claim 11, wherein the oxazaphosphorine payload metabolite is isophosphoramide mustard or its analogs.

25. The composition of claim 11, wherein the oxazaphosphorine payload is selected from dimethyl-isophosphoramide mustard or its analogs or a 4-hydroxy-derivative (4-HO-ifosfamide) or its analog or derivative.

26. The composition of claim 11, wherein the oxazaphosphorine payload metabolite is selected from bromo-isophosphoramide mustard or its analog or derivative comprising evofosfamide or dimethyl-isophosphoramide mustard.

27. The composition of claim 11, wherein the oxazaphosphorine payload is geranyloxy-isophosphoramide mustard metabolite or its analog or derivative.

28. The composition of claim 11, wherein the oxazaphosphorine payload metabolite is mafosfamide or its analog or derivative.

29. The composition of claim 11, wherein the oxazaphosphorine payload metabolite is glufosfamide or its analog or derivative.

30. The composition of claim 11, wherein the oxazaphosphorine payload metabolite is triphosphoramide mustard or its analog or derivative.

31. The composition of claim 1, further comprising a therapeutic agent.

32. The composition of claim 1, further comprising an anti-TAM (tumor-associated macrophage) drug.

33. The composition of claim 1, further comprising one or more antibodies that enhance anti-tumor immunity selected from the group anti-PD-1, anti-PD-L1, anti-CTLA4, anti-LAG3, anti-GITR, anti-TIM-3, anti-TIGIT, anti-CD96, anti-CD226, anti-CD155, anti-CD47, anti-CEACAMI, anti-CEACAM5, anti-CEACAM6, anti-galectin-1, anti-claudin, anti-Siglec-15 antibodies, anti-VISTA, anti-CD137, anti-CCR4 antibody, anti-CCR8 antibody, anti-CD39 antibody, anti-CD25 antibody, anti-CD-73 antibody, and anti-CSFR1.

34. The composition of claim 1, further comprising one or more cytokines selected from IL-1β, IL-2, IL-6, IL-7, IL-12, IL-15, IL-21, IL-23, IL-27, TNFα, IFNα, IFNγ, GM-CSF, anti-IL2R, activators of Toll-like receptors (TLRs), and stimulators of interferon genes (STING).

35. The composition of claim 34, wherein the activators of Toll-like receptors are poly(I: C) and CpG.

36. The composition of claim 1, further comprising one or more chemotherapeutic drugs selected from the group 5-fluorouracil, 2′-deoxy-5-fluoridine, cytarabine, cladribine, fludarabine, pentostatine, gemcitabine, and 6-thioguanine, melphalan and any derivatives thereof; and an alkylating drug chlorambucil, bendamustine, melphalan, alkylating agents or anthracyclines such as doxorubicin, epirubicin, daunarubicin; temozolomide, oxaliplatin, cisplatin, chlorambucil, mechlorethamine, mitoxantrone, pexidartinib, lenvatinib, trabectedin, HDAC inhibitors, anti-angiogenic drugs, bisphosphonates, taxane, vinorelbine, ibrutinib, eribulin, resiquimod, gardiquimod or their analogs, anti-semaphorin 4D, CXCR2 blockers, axitinib, sorafenib, carbozantinib, sunitinib, regorafenib, thalidomide, lenalidomide, pomalidomide, avadomide, vandetanib, cediranib or their analogs, anti-VEGF-A antibody (bevacizumab), anti-VEGF-R2 antibody (ramucirumab), TRL9 agonists, anti-CCR4 antibody, PPARγ agonists, miRNA, angiotensin receptor blockers, CXCR4 blockers, CD4/6 inhibitors, proteosome inhibitors, JAK1/2 inhibitors, Bruton Kinase (BTK) inhibitors, kinase inhibitors, topoisomerase inhibitors, epigenetic inhibitors, DNMT, HMT, HDM inhibitors, PARP-inhibitors, hormone antagonists, anti-prolactin, VEGI, osteopontin, maspin, canstatin, itraconazole, carboxyamidotriazole, suramin, thrombospondin, tetrathiomolybdate, linomide, tasquinimod, carfilzomib, sunitinib, pazobanib, everolimus; anti-hormones: luteinizing hormone releasing hormone (LHRH) antagonists, tamoxifen, cortisol analogs, steroid receptor modulators or antagonists, cancer metabolism inhibitors, radioisotope, radiopharmaceutical, vinca alkaloids, mTOR inhibitors, MEK-inhibitors, BRAF inhibitors, MAPK and tyrosine kinase inhibitors, bortezomib, demethylating agent, bleomycin, alkylating agent, dacarbazine, temozolomide, CELLMODs and targeted protein degrader.

37. The composition of claim 36, wherein the taxane is selected from the group consisting of docetaxel, paclitaxel, cabazitaxel, and 6-α-hydroxypaclitaxel.

38. The composition of claim 36, wherein the epigenetic inhibitor is directed to HDAC, DNMT, LSD1, DOTIL, BET, or EZH.

39. The composition of claim 36, wherein the hormone antagonist is lupron.

40. The composition of claim 36, wherein the PARP-inhibitors are selected from the group consisting of 1-aminobenzamide, iniparib, BMN-573, olaparib, niraparib, talazoparib, rucaparib, veliparib, CEP 9722, MK 4827, BGB-290, and derivatives thereof.

41. The composition of claim 36, wherein the cortisol analogs are selected from the group consisting of predisone, dexamethasone; raloxifene, anastrozole, letrozole, exemestane, spironolactone, cyproterone acetate, bicalutamide, RU53063, thiohydantoin, RD162, and any of their derivatives thereof.

42. The composition of claim 36, wherein the steroid receptor modulators or antagonists are selected from the group consisting of anti-estrogens, anti-progestins, anti-androgens, anti-corticoids, and anti-thyroid hormone.

43. The composition of claim 36, wherein the cancer metabolism inhibitors are selected from the group consisting of pyruvate kinase inhibitors and isocitrate dehydrogenase inhibitors.

44. The composition of claim 36, wherein the radioisotope is radium Ra 223 dichloride, lutetium Lu 177, Actinium 225, Yttrium 90, Technetium 99, or iodine 131.

45. The composition of claim 36, wherein the vinca alkaloids are selected from the group consisting of vinblastine, vincristine, vindesine, and vinorelbine, and any derivatives thereof.

46. The composition of claim 36, wherein the protein degrader is a proteolysis-targeting chimeras (PROTACs) or a molecular glue.

47. The composition of claim 1, further comprising a tumor targeting antibody.

48. The composition of claim 1, further comprising a cell therapy.

49. The composition of claim 1, further comprising a gene therapy.

50. The composition of claim 1, further comprising a cancer vaccine.

51. The composition of claim 1, further comprising an oncolytic virus.

52. A method for treating cancer in a subject in need thereof comprising administering to the subject a composition comprising an antibody-drug conjugate of the formula Ab-(L-D)ₙ wherein
Ab is an antibody, antibody fragment, antibody chain, affibody, aptamer, or a nanobody;
D is an active oxazaphosphorine payload;
L is a linker; and
n has a value of 2 to 20.

53. The method for treating cancer of claim 52 wherein the antibody is trastuzumab.

54. The method for treating cancer of claim 52 wherein the antibody is sacituzumab.

55. The method for treating cancer of claim 52 wherein the linker and active oxazaphosphorine payload is RTX5007.

56. The method for treating cancer of claim 52 wherein the antibody-drug conjugate is trastuzumab-RTX5007.

57. The method for treating cancer of claim 52 wherein the antibody-drug conjugate is sacituzumab-RTX5007.

58. The method for treating cancer of claim 52 wherein the oxazaphosphorine payload is phosphoramide mustard.

59. The method of claim 52, wherein the antibody-drug conjugate is present in a pharmaceutical composition comprising one or more pharmaceutically acceptable carriers.

60. The method of claim 52, further comprising administering a therapeutic agent.

61. The method of claim 52, further comprising administering chemoradiation.

62. The method of claim 52, further comprising administering an anti-TAM (tumor-associated macrophage) drug.

63. The method of claim 52, further comprising administering one or more antibody that enhances anti-tumor immunity selected from the group anti-PD-1, anti-PD-L1, anti-CTLA4, anti-LAG3, anti-GITR, anti-TIM-3, anti-TIGIT, anti-CD96, anti-CD226, anti-CD155, anti-CD47, anti-CEACAMI, anti-CEACAM5, anti-CEACAM6, anti-galectin-1, anti-Siglec-15 antibodies, anti-VISTA, anti-CD137, anti-CCR4 antibody, anti-CCR8 antibody, anti-CD39 antibody, anti-CD25 antibody, anti-CD-73 antibody, and anti-CSFR1.

64. The method of claim 52, further comprising administering one or more cytokines from the group IL-1β, IL-2, IL-6, IL-7, IL-12, IL-15, IL-21, IL-23, IL-27, TNFα, IFNα, IFNγ, GM-CSF), anti-IL2R, activators of Toll-like receptors (TLRs), and stimulators of interferon genes (STING).

65. The method of claim 64, wherein the activators of Toll-like receptors are poly(I: C) and CpG.

66. The method of claim 52, further comprising one or more chemotherapeutic drugs selected from the group 5-fluorouracil, 2′-deoxy-5-fluoridine, cytarabine, cladribine, fludarabine, pentostatine, gemcitabine, and 6-thioguanine, melphalan and any derivatives thereof; and an alkylating drug chlorambucil, bendamustine, melphalan, alkylating agents or anthracyclines such as doxorubicin, epirubicin, daunarubicin; temozolomide, melphalan, oxaliplatin, cisplatin, chlorambucil, mechlorethamine, mitoxantrone, pexidartinib, lenvatinib, trabectedin, HDAC inhibitors, anti-angiogenic drugs, bisphosphonates, taxane, vinorelbine, ibrutinib, eribulin, resiquimod, gardiquimod or their analogs, anti-VISTA antibody, anti-semaphorin 4D, CXCR2 blockers, axitinib, sorafenib, carbozantinib, sunitinib, regorafenib, thalidomide, lenalidomide, pomalidomide, avadomide, vandetanib, cediranib or their analogs, anti-VEGF-A antibody (bevacizumab), anti-VEGF-R2 antibody (ramucirumab), TRL9 agonists, PPARγ agonists, miRNA, angiotensin receptor blockers, CXCR4 blockers, CD4/6 inhibitors, proteosome inhibitors, JAK1/2 inhibitors, Bruton Kinase (BTK) inhibitors, kinase inhibitors, topoisomerase inhibitors, epigenetic inhibitors, DNMT, HMT, HDM inhibitors, PARP-inhibitors, hormone antagonists, anti-prolactin, VEGI, osteopontin, maspin, canstatin, itraconazole, carboxyamidotriazole, suramin, thrombospondin, tetrathiomolybdate, linomide, tasquinimod, carfilzomib, sunitinib, pazobanib, everolimus; anti-hormones: luteinizing hormone releasing hormone (LHRH) antagonists, tamoxifen, cortisol analogs, steroid receptor modulators or antagonists, cancer metabolism inhibitors, radioisotope, radiopharmaceutical, vinca alkaloids, mTOR inhibitors, MEK-inhibitors, BRAF inhibitors, MAPK and tyrosine kinase inhibitors, bortezomib, demethylating agent, bleomycin, alkylating agent, dacarbazine, temozolomide, and CELLMODs.

67. The method of claim 66, wherein the taxane is selected from the group docetaxel, paclitaxel, cabazitaxel, and 6-α-hydroxypaclitaxel.

68. The method of claim 66, wherein the epigenetic inhibitor is directed to HDAC, DNMT, LSD1, DOTIL, BET, or EZH.

69. The method of claim 66, wherein the hormone antagonist is lupron.

70. The method of claim 66, wherein the PARP-inhibitors are selected from 1-aminobenzamide, iniparib, BMN-573, olaparib, niraparib, talazoparib, rucaparib, veliparib, CEP 9722, MK 4827, BGB-290, and derivatives thereof.

71. The method of claim 66, wherein the cortisol analogs are selected from the group predisone, dexamethasone; raloxifene, anastrozole, letrozole, exemestane, spironolactone, cyproterone acetate, bicalutamide, RU53063, thiohydantoin, RD162, and any of their derivatives thereof.

72. The method of claim 66, wherein the steroid receptor modulators or antagonists are selected from the group consisting anti-estrogens, anti-progestins, anti-androgens, anti-corticoids, and anti-thyroid hormone.

73. The method of claim 66, wherein the cancer metabolism inhibitors are selected from the group consisting of pyruvate kinase inhibitors and isocitrate dehydrogenase inhibitors.

74. The method of claim 66, wherein the radioisotope is radium Ra 223 dichloride, lutetium Lu 177, Actinium 225, Yttrium 90, Technetium 99, or iodine 131.

75. The method of claim 66, wherein the vinca alkaloids are selected from the group consisting of vinblastine, vincristine, vindesine, and vinorelbine, and any derivatives thereof.

76. The method of claim 52, further comprising administering a tumor targeting antibody.

77. The method of claim 52, further comprising administering a cell therapy.

78. The method of claim 52, further comprising administering a gene therapy.

79. The method of claim 52, further comprising administering a cancer vaccine.

80. The method of claim 52, further comprising administering radiotherapy.

81. The method of claim 52, further comprising administering phototherapy.

82. The method of claim 52, further comprising administering an oncolytic virus.
